(19) United States
(12) Patent Application Publication
(10) Pub. No.: US 2006/0150989 A1
(43) Pub. Date: Jul. 13, 2006

(54) Method of diagnosing, treating and educating individuals with and/or about depression

(76) Inventor: Peter Migaly, Blairsville, PA (US)

Correspondence Address: THE WEBB LAW FIRM, P.C., 700 KOPPERS BUILDING, 436 SEVENTH AVENUE, PITTSBURGH, PA, 15219, US

(21) Appl. No.: 11/034,447

(22) Filed: Jan. 12, 2005

(51) Int. Cl. A61B 19/00

(52) U.S. Cl. 128/898

(57) ABSTRACT

The present invention relates to methods of correcting misconceptions in the DSM, which can affect improved diagnosis and testing for depression, resulting in better patient satisfaction and global improvement. Also provided are methods comprising administering various medications to produce a “pseudo-placebo” effect in depression, which can guide in selecting a particular treatment method for a more effective antidepressant effect. Another aspect of the invention relates to clinical neuroplasticity in depression by providing a method that increases patient compliance with medication, decreases the bias or prejudice in the public against depression/mental illness, decreases the percentage of treatment resistant depression, decreases patients' resistance and inappropriate use of less effective treatment or treatment without medication because of the existing misperception. Similar methods also can be used successfully for other conditions where depressive symptoms are often present as coexisting condition, such as nicotine addiction, smoking cessation, overweight/weight control and pain management.

### BACKGROUND OF THE INVENTION

1. Field of the Invention

The present invention relates to a new method of diagnosing, monitoring and testing of depressive symptoms that contrasts to the Diagnostic and Statistical Manual IV. The present invention more particularly relates to treating and comparing antidepressive drugs, treatments and combinations thereof in view of the “pseudo-placebo” phenomenon, and extends to new business methods, particularly with respect to education and marketing of these new drugs, treatments and combinations thereof.

2. Background of the invention

Depression affects over seventeen million Americans every year with an estimated cost of over $50 billion dollars annually, which includes expenses related to health care ($12 billion), suicide, and lost productivity at work. Eighty-five percent of depression is related to major depression. In addition, the rate of depression has been rising over the years.

Depression is complicated by the fact that diagnosis and treatment of depression is problematic, in that depressed patients may not seek treatment, the diagnosis of depression may be missed, and when treatment is prescribed patients often fail to comply with their medication or discontinue treatment without informing their doctor. Part of the problem surrounding depression results from people being afraid of the prejudice of mental illness and depression. Indeed, twenty percent of pharmacological resistance is due to patient non-compliance (Thase, M.: 2002 (b), and it's Cit.#11.). One survey has revealed that about one third of respondents reported experiencing symptoms related to depression, however, only eighteen percent of the symptomatic group had ever been diagnosed. (Wallenstein G. V. et al., 2004.)

Additionally, parental depression is known to have a serious impact upon the offspring of depressed parents, depression can destroy marriages and friendships, and robs people of a fulfilling life.

Further, depression carries the risk of suicide. In the United States, suicide accounts for approximately 30,000-35,000 deaths a year, numbers similar to those leukemia as a cause of death. Suicide has a profound long term effect on family members, friends and coworkers that are left behind, in that there is a very complicated form of bereavement. Suicide also has a marked impact upon therapists other members of the treatment team.

Also recognized, especially in children and adolescents suffering from depression, is the fact that many studies performed to test the efficacy of antidepressant medications fail to differentiate the effects of the medications from a placebo effect. Thus, beneficial antidepressant medications are missed due to the failure to differentiate them from a placebo effect. Current methods for testing antidepressant medications do not make it economically feasible to assess which antidepressants are the most effective.

Therefore, because current methods for diagnosing, treating, and monitoring patients with depression are flawed and problematic, there exists a need for better diagnostic, treatment and monitoring of patients with depression, as well as methods for educating patients, health care workers and the general public about depression, using marketing techniques.

### SUMMARY OF THE INVENTION

The present invention provides methods of diagnosing and treating a depressive disorder in a patient, which includes challenging the traditional dogma of the Diagnostic and Statistical Manual IV (DSM-IV), which has set the current standard of care with it's criteria sets, as being only a differential diagnostic manual primarily useful for differentiating depression from other mental disorders; challenging that the DSM IV's limited number of depressive symptoms in it's criteria set is accurate and sensitive enough to diagnose, monitor and test for depressive symptoms and accurate and sensitive enough to test for remission of depressive symptoms; and challenging that the Hamilton Depression Rating Scale, which rating scale relies on the Diagnostic and Statistical Manual IV's limited number of depressive symptom list, is accurate and sensitive enough to diagnose, monitor and test for depressive symptoms and accurate and sensitive enough to test for remission of depressive symptoms. The limited number of depressive symptoms provided in the DSM-IV consists of decreased sleep, decreased interest, guilt, decreased energy, decreased concentration, decreased appetite, psychomotor retardation and suicidal ideation. The methods of the present invention are achieved by providing an extended list of depressive symptoms that is accurate and sensitive enough and that can be systematically relied upon to diagnose, test and monitor for depressive symptoms. The extended list includes, without limitation, anxiety, somatic concems/somatization, focusing on somatic symptoms, rumination/obsessiveness, anger outbursts/impulsivity/hostility/violence, cognitive distortion/global thinking, cognitive deficit/impairment, social withdrawal, helplessness/hopelessness, acute and chronic stressors and the patient's ability to cope with them as well as to problem solve, perception of unjust and resentment, indifference, sensitivity, and other symptoms and observed signs. We define systematic reliance and/or application of these symptoms, that if possible most or all of this extended symptoms should be utilized, and not just random selection from this list.

The present invention also provides methods for producing a pseudo-placebo effect in a patient afflicted with a depressive disorder which results in conditioning and expectation changes in the patient that are relied on and utilized to treat the depression in the patient. The pseudo-placebo effect includes targeting each of the depressive symptoms, which include the extended symptom list, and not only the general depressed mood itself, with psychological and/or pharmacological intervention. The methods of the present invention therefore increase the effectiveness of the treatment of depression, provide a quicker response to treatment, provide a better chance to achieve full remission and increase a patients' quality of life, and decreases the risk of suicide. Furthermore, the percentage of cases of treatment resistance depression and percentage of cases of depression only in partial remission are decreased in the population of patients afflicted with depression.

Examples of the pseudo-placebo effect utilizing a pharmacological treatment include, without limitation, antidepressants, such as, without limitation, serotonin reuptake inhibitors, selective norepinephrine reuptake inhibitors, combined action SSRI/SNRI, serotonin-2 antagonist/reuptake inhibitors, antidepressants with alpha-2 antagonism plus serotonin-2 and serotonin-3 antagonism, antidepressants with serotonin/norepinephrine/dopamine reuptake inhibition or antidepressants with norepinephrine and dopamine reuptake inhibition. Additionally, antidepressants can include, without limitation, tricyclic antidepressants, tetracyclic antidepressants, MAOI inhibitors, clomipramine, fluoxetine, norfluoxetine, paroxetine, sertraline, fluvoxamine, citalopram, escitalopram, bupropion, nefazodone, mirtazapine, venlafaxine, duloxetine, milnacipran, reboxetine, zimelidine, indalpine, gepirone, femoxetine, alaproclate, duloxetine or pharmaceutically acceptable salts thereof. Other pharmacologic agents can include, without limitation, 5-HT-1 alpha antagonists, 5-HT-1beta antagonists, 5-HT1A receptor agonists, 5-HT1A receptor agonists and antagonists, 5-HT2 receptor antagonists, viloxazine hydrochloride, dehydroepiandosterone, NMDA receptor antagonists, AMPA receptor potentiators, substance P antagonists/neurokinin-1 receptor antagonists, nonpeptide Substance P antagonists, neurokinin 2 antagonists, neurokinin 3 antagonists, corticotropin-releasing factor receptor antagonists, antiglucocorticoid medications, glucocorticoid receptor antagonists, cortisol blocking agents, nitric oxide synthesize inhibitors, inhibitors of phosphodiesterase, enkephalinase inhibitors, GABA-A receptor agonists, free radical trapping agents, atypical MAOI's, selective MAOI inhibitors, hormones, folinic acid, leucovorin, tramadol, tryptophan, pharmaceutically acceptable salts thereof or combinations thereof.

Examples of the pseudo-placebo effect utilizing a pharmacological treatment can include, without limitation, atypical antipsychotics and/or dopamine system stabilizers, and/or antipsychotics selected from the group consisting of risperidone, quetiapene, olanzapine, ziprasidone and aripiprazole, wherein said atypical antipsychotics can include, without limitation, olanzapine, iloperidone, Org 5222, melperone, amperozide, SM-9018, JL-13, perphenazine, trifluoperazine, zotepine, flupenthixol, amisulpride, and sulpiride, pharmaceutically acceptable salts thereof, or combinations thereof.

Examples of the pseudo-placebo effect utilizing a pharmacological treatment can include, without limitation, anxiolytics, such as, without limitation, clonazepam, benzodiazepines, antipsychotics, atypical antipsychotics dopamine system stabilizers or combinations thereof.

Examples of the pseudo-placebo effect utilizing a pharmacological treatment can include, without limitation, targeting decreased or disturbed sleep with sleeping pills, such as, without limitation, zolpidem and benzodiazepines, as well as targeting sleep problems through sleep hygiene, correcting sleep apnea with continuous positive airway pressure (CPAP), or correcting overweight.

Examples of the pseudo-placebo effect utilizing a pharmacological treatment also can include utilizing a pharmacological treatment that includes medications that target anger/violence/anxiety, such as, without limitation, the beta blockers propranolol or pindolol.

Further examples of the pseudo-placebo effect utilizing a pharmacological treatment can include, without limitation, medications targeting fatigue and tiredness, such as modafinil, medications targeting decreased energy, such as stimulants, or medications that target disorders such as, without limitation, obsessiveness/rumination, cognitive distortions, jumping to conclusion, somatic symptoms or perceptual disturbance relating to somatic symptoms. Examples of the pseudo-placebo effect utilizing a pharmacological treatment can include any of the pharmacological treatments or combinations thereof that are known to target these symptoms.

The methods of the present invention encompass psychological testing via psychometric instruments that incorporate and systematically rely upon the extended symptom list. The number of extended symptoms contained in the psychometric instruments can range from about at least five extended symptoms to about eight extended symptoms or more.

Psychometric instruments that are based on the more extended symptom list can provide a more sensitive method to test and monitor depression than existing tests and can provide a more practical way to better test antidepressant effectiveness and to test which antidepressant or combination thereof is more effective and/or quicker acting.

The present invention also provides methods of increasing treatment adherence and decreasing resistance for seeking help in a depressed patient, as well as decreasing or eliminating prejudice and the stigma against depression as a mental illness, by describing to the patient, as well as to health care providers and to the general public, about clinical studies that do not pertain directly to mental depression but that involve neuroplasticity of the brain, neurogenesis, and/or brain mapping. Such descriptions of clinical studies unrelated to depression are a metaphor that the patient, health care provider or the general public can associate, or link, to neuroplasticity in the brain and mental depression and/or brain mapping in depression.

The description of clinical studies also can include, without limitation, as if/role play experiments shown to cause depression or to lift depression and/or environmental situations shown to cause depression in everyone, whereby the resolution of these environmental situations or as if/role play experiments can be linked to the resolution of depressive symptoms with its accompanied neuroplasticity.

The general term “neuroplasticity” includes the term “clinical neuroplasticity.” As a term as used herein to distinguish the term neuroplasticity (an adaptation of the brain/or nerves to changes), or synaptic plasticity (changes observed at molecular or subcellular level) from “clinical neuroplasticity” to meet the definition of “clinical neuroplasticity” correlating changes in sensations/motor functions/emotions need to be mapped in the brain (not just simply saying that there is a “neurogenerative” process or new neurogenesis observed; and these changes in the brain should also be linked to a well explained functional neuroanatomy, (an understanding of the correlation of a change in that brain area with the function of that brain area) and therefore allow to explain this phenomena to the average clinician, and or the public so that it would be easy to understand.

Such descriptions of clinical studies unrelated to depression that are a metaphor for the patient, health care provider or the general public can consist of, without limitation, the metaphor of an “invisible mitt” being equivalent to restraining negative thought patterns/rumination with medications and/or with cognitive therapy, the metaphor of practice or practice makes a master, i.e., practicing the equivalent of turning of dominos in the physical therapy of stroke victims, which is equivalent to the practice of cognitive therapy; or the metaphor of the need for practicing having optimistic thoughts, which can result from increasing positive expectations. The present invention also provides a description and examples of pseudo-placebo conditioning/expectation changing effects of medications on depression and gives explanation of why antidepressants have a large placebo effect.

Additionally, describing metaphor to a patient can be used to explain the risk of relapse to depression if medications are discontinued, or if patterns of thoughts are shifted again from predominantly positive to negative/ruminative thoughts.

The methods of the present invention also include linking for the patient, health care providers and to the general public that the hippocampal volumetric and/or morphologic changes seen in depression may result from the process of learning and/or memory, as learning is involved in the process of mental depression, rather than resulting from the change of mood per se. This new way of understanding the reasons for hippocampal changes in depression therefore fits in well with findings that the hippocampus is involved in learning and memory, which may guide pre-clinical research for new antidepressants.

The present invention further provides methods of increasing treatment adherence and decreasing resistance for seeking help in a depressed patient by educating the patient, health care providers and the general public, via various marketing techniques, that meeting the patient's needs by the healthcare provider, and for the healthcare provider to repeatedly change his or her approach so that the patient's needs are met, a desired change can occur. By doing so, the patient perceives that the situation is right and thus has a readiness for change. The provider needs to change his or her approach so that the patient also can perceive that the timing for change is the right time and that would be an overwhelming benefit to change that the patient is able to achieve, that the change is important and emergent enough, that the relative easiness of the change or at least some enjoyment for the change can be realized, and the healthcare provider to change his or her approach to assure that the patient they has the tools and skills needed for such desired change. This method also includes describing to the patient new information in simple ways so as to make the patient feel as if they already know the information, thus relating to the information and/or accepting the information as their own. This method also elicits positive emotions and expectations from the patient, validates the patient's feelings through universal human experiences, raises the hope and/or increases confidence in the patient, gives the patient new, direct, personal experience that change is possible, and acknowledges that rapid change is possible.

Using the methods of the present invention, the effectiveness of treatment of depression is increased, and protects against or remedies the development of tolerance towards antidepressant treatment. Further, the methods of the present invention prevents a paradoxical effect of the antidepressant treatment that sensitizes patients to depression, avoids or treats worsening of depression from the antidepressant treatment, treats residual symptoms of depression, and/or decreases suicide and/or the risk of suicide.

In some of the preferred embodiments the methods of the present invention are given as initial treatment, as soon as practicable, or upon presentation to a physician or a health care provider for preventing suicide.

It is envisioned that the methods of the present invention can be used in cases other than depression, such as diagnostic categories where depression is often a co-morbid and/or underlying condition, such as, without limitation, smoking cessation, nicotine withdrawal, addiction, overweight/obesity/weight control, eating disorders, chronic pain, other mental disorders, or in other cases not related to depression.

### DESCRIPTION OF THE PREFERRED EMBODIMENTS

The present invention provides methods for eliminating impediments and increasing the effectiveness of the diagnosis, testing/screening and treatment for depression. The methods include improved educational and or marketing techniques that eliminates or reduces prejudice against mental illness/depression, improves medication/and or treatment adherence, and/or decreases the number of untreated depression, for example by using a “clinical neuroplasticity” metaphor. The methods can be used to identify issues and solutions to new drug development and testing, including placebo and “pseudo placebo” effects, and/or can provide more practical ways to better test antidepressant effectiveness, and/or to test that which antidepressant or combination is more effective and/or quicker acting, and/or can be used to scrutinize and/or critique existing or future studies, including those sent for new FDA approval. All of the methods provided herein can also increase the effectiveness of the treatment of depression, including treatment resistant depression. While these methods are invaluable by themselves, the combined utilization of them can further enhance the goal of decreasing a patient's suffering from depression.

The Diagnostic and Statistical Manual IV-TR., 2000 (DSM-IV) and similar nomenclatures, such as ICD-10 (Kaplan, H. I. et al. 1998), have been criticized before, but a major flaw that they contain has been missed, which the present invention corrects. This will be described by using the example of depression, primarily major depressive disorder (MDD).

While it has correctly been recognized that individuals sharing a diagnosis, such as depression, are likely to be heterogenous in the defining features of the diagnosis (DSM IV-TR p xxxi), and that there is a long list of possible symptoms under which depression in different severities can present, there is a misconception in DSM-IV, namely, that individuals only need to present with a subset of items from a longer list of criteria sets for the diagnosis (See e.g. DSM IV-TR p xxxii and 356, 375-376), therefore flawing the sensitivity of the diagnosis.

According to the DSM-IV, the diagnosis of MDD requires the presence of a major depressive episode (a building block to diagnose MDD and other mood disorders, such as bipolar disorder). This in turn consists of at least five of the nine symptoms present during the same two-week period, of which depressed mood or loss of interest or pleasure needs to be present as one of the symptoms. Changes in weight/appetite, sleep, energy, psychomotor retardation or agitation, guilt, decreased concentration and suicidality are the other symptoms. One does not need to have all of the symptoms present for the diagnosis, and MDD or a major depressive episode therefore is not equal to the individual symptoms, as some may be absent. Additionally, there are some exclusion criteria for both MDD and major depressive episodes.

The misconception of the DSM with respect to depression is that instead of being a Diagnostic and Statistical Manual, it would be more appropriate to call it a Differential Diagnostic Manual. This is because, instead of a diagnosis, DSM provides sets of criteria to distinguish depression from other disorders, like anxiety, somatophorm disorder, or obsession, in which the sets of critera artificially create a specific disorder. For the purpose of differentiating depression from other mental illnesses, DSM is an excellent resource. However, the DSM criteria sets completely fail when it comes to diagnosing and monitoring diagnostic symptoms during or after treatment to determine if the treatment is effective, if there is a remission or symptoms, or if there is a relapse of symptoms. Thus, the DSM falls short of what it is supposed to do, namely diagnose and monitor/test symptoms for depression. This failure is the result of not listing the appropriate amount of depression symptoms in the criterion part. Nevertheless, the DSM currently is in the mainstream for diagnosing depression.

This present invention utilizes the DSM for its criteria set of depression (e.g. major depressive disorder, or MDD) in order to differentiate it from other mental disorders. However, once depression is diagnosed with the DSM (or with similar nomenclatures like ICD-10), the methods of the present invention utilize an “extended symptom list,” which allows for a more precise diagnosis, a more precise assessment of the depression's characteristics and severity, and allows for the monitoring of the patient with respect to improvement or relapse of depression. Because the methods of the present invention are more sensitive, they can be used to decide which antidepressant or medication combination is more effective, quicker acting, or show a particular benefit. Additionally, with the use of the extended symptom list, treatment decisions can be made that were not possible with the DSM, such as guidance for the use of a particular antidepressant or other medication, use of adjunct medications, or starting treatment with a particular medication or adjunct medication. Furthermore, the realization that various medications have a “pseudo-placebo” effect on depression, as described below, further stresses the importance of the use of the extended symptom list of the present invention in selecting a particular treatment method in order to achieve a more effective antidepressant effect.

The methods of the present invention also can be applied in other mental health disorders.

Depression consists of a cluster of symptoms. This is reflected in the clinically used acronym provided by the DSM, “SIGECAPS,” in which S=decreased sleep, I=decreased interest, G=guilt, E=decreased energy, C=decreased concentration, A=decreased appetite, P=psychomotor retardation and S=suicidal ideation. (For an overview of the depressive symptoms and the mnemonic “SIGECAPS” see Carlat, D. J. 1998).

However nomenclatures like DSM are artificially created definitions. These definitions help psychiatrisst to distinguish between anxiety disorders, depressive disorders, and thought disorders, such as psychosis. However, looking at the currently used depressive symptoms, such as a decrease in appetite or sleeplessness, one needs to ask how often do they occur and whether it really matters for purposes of making a diagnosis if these symptoms are not present. According to the DSM, one needs only five symptoms to make a diagnosis of depression. However, the frequently seen symptom in depression of anxiety is not required for diagnosis. This is because the DSM has artificially created another category for those anxiety disorders, referred to as a “comorbid disorder”. In fact, it should be noted that anxiety is frequently more present in depression than many other “depressive symptoms,” but it is not included in diagnosing and treating depression. The same is true for other symptoms frequently present in depression that currently is not included in diagnosing depression, such as rumination, in which another category is created, referred to as the “OCD spectrum of disorders.” Additionally, other mental illnesses, such as schizophrenia, also can present with OCD symptoms.

Thus, depression is a mixture of many other symptoms that currently is not included in making the diagnosis, as well as being disregarded in treating the illness.

Therefore, the present invention provides an improved definition, diagnosis, and treatment of depression, such as MDD, which includes the following symptoms:

Anxiety, including somatic concems/somatization, focusing on somatic symptoms. {A} {S}

Rumination (OCD symptom) (and focusing on negatives)—overlaps with rumination for guilt. (R}

Anger outbursts/impulsivity/hostility/violence, and resentments. (This is more than suicidal thoughts/acts). {AHIV}

Cognitive distortion/global thinking. (Cognitive therapists have recognized this symptom is depression, but it is still not used in the diagnosis, routine assessment, and, most importantly, in medical treatment). Cognitive deficit/impairment may be a related phenomena, which is not exactly the same as “diminished ability to think.” This category is different from social withdrawal, or a decrease of interest). {CD/CD}

Social withdrawal, which is different from a decrease of interest, or cognitive impairment/distortion. {W}

Helplessness/hopelessness. This category is known but not included in the DSM-IV-TR. {H/H}

Acute and chronic stressors, and the patient's ability to cope with them and to “problem solve”. (In the search of new antidepressants, it has been recognized that stress and cortisol levels may play a role in depression, but this category has not been included in diagnosis or testing. {S}

Other symptoms and observed signs {O/O}: This includes a number of other symptoms that should be monitored and addressed.

Of academic and clinical interest, social withdrawal may come not only from a depressed mood, but from the “misreading of others emotions,” which overlaps with cognitive distortion, as well as a decrease of “bidding for attention” that is found in healthy relationships, i.e., reacting to other peoples statements and similarly inviting them from time to time to interact with you. Thus, the “misreading of others emotions” and a decrease of “bidding for attention” contributes to relationship conflicts and produces further stress and withdrawal.

Other symptoms, such as apathy, lack of feelings, affective unresponsiveness, also can be included in the diagnosis, treatment and monitoring methods of the present invention. Also important are unchanging facial expressions; lack of verbal inflictions, i.e., monotone speech, poverty of speech or content of speech; decreased spontaneous movement, i.e., decreased gestures; poor eye contact; increased latency to respond; and thought blocking. Other signs of depression are difficulty reading, sustaining a conversation and collecting one's thoughts, as well as difficulty falling asleep, or waking up at the middle of the night, which may also be related to rumination, i.e., automatic thoughts. Other notable symptoms are lack of harmony and conflicting feelings.

It is interesting to note that in raising children, future oriented talk that is positive and optimistic, predominates, whereas in depressed patient's the thought content typically is negativistic, with rumination about the past predominating.

Perception of unjust and resentment, with frequent, intrusive thoughts relating to this, also may be a symptom of depression. {U, R} This may be due, in part, because of a decrease in ego boundary accompanied with the increased exposure and vividly described and shown violent news, as well as an unjust media. On the other hand, indifference {I,} and numbness, as seen in post traumatic stress disorder (PTSD), also may be present in depressed patients. Depressed patients may also tend to take things in life personally and to personalize insults, i.e. be overly sensitive. {S,} (Some of these signs are non-specific to depression, as for example, caring about the world, which is good personality trait. The problem arises when one feels that they cannot do anything about it and it takes a toll on them).

A tendency for quick and impulsive decision making, i.e., shopping, overeating, or forming an opinion, also can be characteristic of depression, which overlaps with the cognitive distortion of “jumping to conclusions.”

In summary, in addition to a depressed mood and “SIGECAPS” (sleep, interest, guilt, energy, concentration, appetite, psychomotor retardation, suicide), the extended list of depressive symptoms of the present invention includes:

“ARS, AHIV, W, H/H, S, CD/CD, S, O/O, URSSI” for the above symptoms (or AHIV AROWS CD/CD H/HORSS IS U).

The methods of the present invention include all of the symptoms of the extended list. Additionally, “O/O,” for other symptoms/observed signs, is left at the discretion of the clinician/researcher to decide whether to select other depressive symptoms, such as listed above or presented by the patient, which causes the functional impairment or distress. Thus, the diagnosis and testing for depression needs to focus on a wide range of symptoms and not just on the diagnostic criteria sets of the DSM.

Suicidality can be reclassified as being “out of touch with reality,” similar to psychosis. This reclassification would justify the use of antipsychotics, as well as mood stabilizers for impulsivity, in the treatment of depression.

Because depression consists of many different symptoms, addressing each and every one can improve treatment and result in a quicker and fuller resolution of depression. This is referred to herein as a targeted or “stepwise approach”. That is, it is necessary to target more than just one depressive symptom, such as mood. Thus, the present invention allows for the use of more than one medication if indicated for symptomatic relief or for their overwhelming benefits, e.g., prevention of suicide (SI).

The methods of the present invention therefore provide new diagnostic criteria for depression, as well as for suicidality, which includes different treatment approaches with correspondingly different psychological testing.

One problem that has been identified with the current depression testing instruments is that a large number of patients are needed for the tests to differentiate between the effectiveness of the antidepressants, which is economically impractical. Thus, the development of new psychological testing scales based on the extended symptom list of the present invention, overcomes this problem. New tests can be developed that are more precise in assessing even subtle differences in the efficacy of antidepressants, the differences between antidepressant medications (which currently are believed to be equally efficacious), or in assessing the differences between a particular antidepressant and a combination therapy. With such psychological testing instruments, a timeline of improvement (e.g. more rapid/earlier improvement) also can be better assessed. Differences in patient symptomatology also can better guide treatment protocols, such as emphasizing the need for initial treatment with a combination treatment protocol.

Any protocol that causes an improvement in these heretofore unlisted diagnostic symptoms of depression would improve the depression, the overall good feelings of the patient and the patient's quality of life. Therefore, the treatment methods of the present invention specifically target the individual depressive symptoms and these heretofore unlisted, more extensive diagnostic symptoms. Such targeting of symptoms enhances treatment response and also reduces the percentage of treatment resistant depression (TRD).

In treating depression the more symptoms one addresses and corrects “right away,” the better the chance of patient satisfaction and global improvement. This is another way in which the more extensive symptom list is invaluable. The methods of the present invention provide for vigorously targeting different depressive symptoms with multiple or adjunct treatment modalities, resulting in a more rapid and effective treatment. This is more particularly described below.

In finding that the addition of adjunct medication results in an almost immediate positive response in depression, one must also look for a psychological explanation. In short, an immediate improvement in any one of a patient's symptoms is a direct reinforcement that change is possible, which changes the patient's expectation. It should be noted that patient expectation also has a major role in the placebo effect. Generally, depressed patients, have low motivation, decreased energy and interest, and thoughts of “why bother,” “nothing is going to change,” “nothing is going to help.” In other words, they have feelings of helplessness and hopelessness. Indeed, helplessness and hopelessness are feelings characteristic of depression, and also are a significant risk factor for suicide. Unfortunately, this is why many depressed people do not seek treatment. It is ironic, but when they go for an evaluation by a doctor, this negative expectation is reinforced. This is because they do not get immediate relief and the evaluating doctor asks many questions concerning painful or negative aspects of their lives. Indeed, at times it seems to them that the doctor is just dwelling on their problems. Typically, at the end of the first visit they are told that antidepressant medication will take several weeks to work. As a result, negative expectations are reinforced, and because of their feelings of hopelessness, they may discontinue taking their medication. Nonadherence to prescribed medication accounts for as many as 20% of the cases considered to be treatment-resistant, and approximately 24% of patients do not inform their physicians that they have stopped taking antidepressants. (Demyttenaere, K. et al. 2001). Other publication reports that in primary care, more than one third of patients fail to refill their initial antidepressant prescription, and nearly half discontinue it within three months (Pincus, H.A., et al. 2001,).

Therefore, addressing and relieving the anxiety which is present as a comorbid disorder in 56.8% of patients with known non-bipolar, major depressive disorder, can result in a drastic change in a patients' expectation of success. Because a positive change has occurred, e.g., relief from anxiety and an improvement in their overall feelings, they would have more hope. Therefore, by pharmacologically addressing one symptom, improvement in other related symptoms, and in the depression generally, can be expected. This explains the subjective feeling of “immediate/rapid” improvement from the psychological point of view, and why one should pay attention to the other depressive symptoms that are omitted from the DSM criteria sets. Thus, the present invention provides methods for the pharmacological improvement in any of the depressive or discomforting symptoms which results in ameliorating another depressive symptom: helplessness and hopelessness.

Sleep disturbances, such as insomnia, is one of the symptoms often present in depression. Addressing this problem early on would similarly result in improved compliance and in a more immediate improvement overall in depressive symptoms. Temporarily adding a sleeping pill, such as like zolpidem (Ambien), until the depressive symptoms, as well as the insomnia, lifts, can therefore have a more beneficial effect than the improvement of sleep per se. It is important to note that neuroleptics, in particular atypical neuroleptics, can improve sleep (Salin-Pascual, R. J. et al. 1999.) This again points to the benefit of combining these medications with antidepressants. Another reason leads us to the same conclusion concerning combining medications, i.e., the antipsychotic-antidepressant combination has been used for treatment resistant obsessive-compulsive disorder (OCD), and if one considers that rumination, one of the extended depressive symptoms, is reduced with the use of these medications, then the sleep disturbance caused by rumination when going to sleep also would be eliminated.

There are stories of the “miracle” effect of using stimulants as antidepressants in some medically ill/elderly patients (Kamholz, B. A., et al. 1996). (See also the reference for stimulant use in the medically ill/elderly: Satel, S. L. et al. 1988). A similar explanation—not being put into this context before—may be involved here, in that by improving a patient's energy, i.e. correcting a depressive symptom, one sees a quicker response than with other antidepressants. With global improvement, the patient's expectation changes as well, and feelings of hopelessness become less pronounced or goes away entirely.

However, in defense of a biological explanation, one needs also to note the following: a high placebo response plays a role in the treatment of depression. The mean placebo response rate for major depressive disorder is about 30-40% with some studies reporting rates of 70% (Schatzberg A. F, et al., 2000). Some studies support that trend, showing that patients with more severe depression respond well to antidepressants whereas those mildly ill respond equally well to antidepressants and placebo (Khan, A., et al., 2002). However, it is unlikely that placebo or “pseudo-placebo” responses would be the only explanation when one targets the various depressive symptoms with combination therapy. Studies specifically emphasize the proposed usefulness of antipsychotics, primarily for the antipsychotic-antidepressant combination treatment of depression. First, the adjunct medication is chosen specifically to target pharmacologically a specific symptom that is related to depression, e.g., anxiety, helplessness and hopelessness, rumination/guilt, cognitive distortions overlapping with psychosis, low energy/tiredness, sleep disturbance, or anger outburst/impulsivity/violence respectively), so the adjunct medication is not a placebo. Adjunct medications in targeting the treatment of depression can be viewed as a non-specific pharmacologically active specifically symptom-specific “pseudo-placebo” (definition provided below). Second, as is reported with the risperidone-SSRI combination for treatment-resistant depression (O'Connor, M., et al 1998), at least in one case it was reported that the patient relapsed despite the resolution of the sleep problem (and despite the additional adjunctive use of a benzodiazepine as an anxiolytic); and it did not have the same result in the improvement of depression as did the added atypical antipsychotic. Therefore, a psychological explanation for the role of changing expectations is extremely important, but it is not the full answer.

Only one medication apparently is used off label based on it's mechanism of eliciting a conditioned reflex, or as used herein a “pseudo-placebo” effect. Propranolol (Inderal), a beta blocker, for example titrated up to a dose of 40 mg four times a day or higher, not only reduces heart rate, but is used for impulse control, i.e., aggressive violence. It works, by keeping the heart rate low even when people get angry and start to act out. This suggests, or gives a “false” feedback to the patient, that they are not in a flight or fight response situation, but rather everything is calm because the heart still is beating at a slow rate. The same principle applies when this medication is given for “stage-fright”/anxiety. This is not a placebo effect, as the medication has a specific pharmacological effect of slowing the heart rate down, and it is specifically selected for that action. However, it achieves it's desired action indirectly, relying on psychological principles like expectation and a conditioning effect. (It is noteworthy that propranolol has the potential side effect of mental depression, which may limit it's usefulness as an adjunct medication for depression).

Pindolol, a non-selective beta blocker, has been studied and used as an adjunct to some antidepressants in order to enhance the antidepressant effect giving a different explanation there. (See, for example, WO 99/58130, May 1998, combining noradrenergic reuptake inhibitors such as reboxetine with pindolol). This is noteworthy because if beta blockers act on anger/impulsivity/anxiety-fear i.e., within the extended depressive symptoms list, then any improved antidepressant effect could support the theory that it was the result of the “pseudo-placebo”/conditional reflex/expectation changing effect, even if it was pharmaceutically targeted—hence, the “pseudo-placebo” effect or expression.

Thus, when one targets depressive symptoms with various medications, as provided in the methods of the present invention, besides having a direct effect on mood, the medications also may elicit a conditioned reflex, which is a reinforcement that change is possible, therefore improving hopelessness/helplessness and the patients' overall good feelings, the mood itself. Similar to the action of propranolol, this is not a placebo, but rather a pseudo-placebo effect, which elicits a conditioned reflex and further change in the patient. The present invention presents this novel phenomenon, which can be a key factor to better separate medication effects from placebo effects in drug development trials.

This new phenomenon also is important because it takes the focus away from neurotransmitters like 5-HT (serotonin) or nor-epinephrine (NE), and stresses the importance of targeting the extended list of depressive symptoms. This phenomenon also can be important for re-evaluating various animal models of depression.

It is known that changes in one neurotransmitter likely affects other neurotransmitters, and that our “simplified neurotransmitter theories” are more complex than once believed, e.g. many if not most neurons release more than one neurotransmitter (Trudeau, L-E. 2004). With the development of selective serotonin reuptake inhibitors (SSRIs), the role of serotonin in depression has been recognized. However, it is possible that it really is NE that is the primary neurotransmitter implicated in depression, i.e. effecting mood per se, with serotonin implicated more in the OCD component of depression, such as rumination and the pessimistic focus on the negatives, and only a consequential secondary effect on mood itself. Serotonin also has a role in learning and memory in the hippocampus. Appreciation of the pseudo-placebo effect can have further implications in clinical practice, patient education, marketing and new drug development. One example of this would be to shift our focus to medications that affect both NE and serotonin or multiple neurotransmitters. Alternatively, two medications, such as a NE agent and a 5-HT agent, i.e., an SSRI, can be combined. Although current psychological tests do not show if one antidepressant is better than the other, there is an opinion by some that “dual action antidepressants” may be better.

Modafinil (Provigil), originally introduced as a wake-promoting agent for excessive daytime sleepiness associated with narcolepsy, may reduce tiredness/fatigue and sleepiness in depressed patients. This is another example that targeting individual depressive symptoms one by one, a more rapid or more pronounced antidepressant effect can be achieved.

Up until now it has been believed that, whereas other medical disorders are named by their etiology, e.g., infection, not fever; diabetes mellitus, not high blood sugar, psychiatry lagged behind by naming disorders according to their symptoms, such as like depression, which has caused confusion. As presented herein, depression, i.e., the depressive disorders, appears best treated by targeting their symptoms and not restricting the treatment and explanation for their cause to a single cause, such as a neurotransmitter deficit or imbalance. The “clinical neuroplasticity” model of depression, described more particularly below, can be a better tool in guiding treatment and research decisions.

By pharmacologically reducing obsessiveness/rumination and/or cognitive distortion, the methods of the present invention also reduce guilt, another depressive symptom. For example, the antipsychotic-antidepressant combination has been used for treatment resistant OCD. Consequently, the sleep disturbance caused by the rumination when going to sleep also is eliminated.

Cognitive distortions, such as jumping to conclusions without analysis of the facts, are characteristic for depression. Cognitive therapy specifically addresses this problem by teaching patients how to recognize and correct these distortions. Cognitive distortions are also referred to as “global thinking,” which has been reported as the thinking style of the depressed. There is, however, an overlap between cognitive distortions, the “mini psychosis” of borderline personality disorder (BPD), and the “full blown psychosis” of psychotics; all of them being out of touch with reality but in different degrees.

Psychiatrists also know that antipsychotics are not particularly effective in patients with chronic delusions of only one delusional idea (monoideatic delusions). Nevertheless, doctors prescribe antipsychotics despite their limited usefulness. (The neuroplasticity model for chronic delusion may explain its relative resistance to medications [Spitzer M. 1999]). In the present invention, it is postulated that the atypical antipsychotics can be useful for depression, thus these medications may, in part, be targeting the cognitive distortions that overlap with psychosis. Indeed, it may be worthwhile to consider reclassifying depression as a “thought disorder,” or at least appreciating that the overlap between depression, i.e., mood disorders, and thought disorders should not be limited to psychosis or psychotic depression per se. (Disorders such as psychosis, schizophrenia and delusional disorders, where the patients are out of touch with reality, are listed under the category of “thought disorders” and are treated with antipsychotic medications). Depression, with the exception of psychotic depression, was and still is not considered a “thought disorder.” However, the particularly strong cognitive distortions in depressed patients, along with impaired reality testing, do overlap with psychosis. This indicates that depression, at least in part, should also be considered a thought disorder. This provides support for the use of antipsychotic-antidepressant medications for the treatment of depression, as provided in the present invention.

Cognitive distortions also play a role in anger attacks that 30-40% of depressed patients display. A significant association between depression and violent behavior in community samples also had been reported (Koh, K. B. et al., 2002). Some reports have shown that 28-44% of depressed outpatients exhibit violent behavior (Hughes, D. H. 1998). Antipsychotics have been used to reduce violence in acute settings, like in emergency rooms, and also have been given to long term to psychotic/bipolar patients. Antipsychotics also have been used for “pathologic aggression.” Therefore, the use of antipsychotics as adjunct medication in the treatment of depression, i.e., major depressive disorder, dysthymia, “double depression, appears warranted.

It has been speculated that jumping to conclusions without analyzing the facts can lead to impulsivity and thus increase the chance for suicide. Indeed, it is known that impaired reality testing, as shown in alcoholism and drug abuse, is associated with a significantly increased risk for suicide. Alcohol is associated with 25 to 50% of all suicide cases and is the second most comorbid factor after depression. Therefore, addressing cognitive distortions, impaired reality testing, and/or the source of impulsivity is essential, and further supports the combination use of psychotropic medications.

Depressed patients, because of their strong cognitive distortions, may not only misperceive information coming from the environment, such as miscommunications in their relationships that lead to social isolation, but also can misperceive stimuli coming from their own body. It is known that depressed patients have increased somatic symptoms (Stahl, S. M. 2002), with the majority of the depressed patients presenting only with physical symptoms to primary care providers. This demonstrates a support for the invention that depression also presents with a perceptual disturbance symptom, in which, just as for delusions, treatment with neuroleptics in combination with antidepressants can be useful. Therefore neuroleptics may be used to target this symptom and improve depression through the “pseudo-placebo” effect.

It is important to reassess the role of cognitive distortions in hopelessness and suicide. The predictive value of hopelessness in suicide has been confirmed, and indeed, hopelessness is the greatest predictor of suicide risk beyond the first year. However suicide occurs in only 5% of terminally ill patients and their greatest risk factor is untreated depression. Therefore it is not hopelessness per se, but its perception, i.e., the cognitive distortion characteristic of depression, which seems to be the most important factor. The adjunctive use of antipsychotics with SSRIs and newer antidepressants in the treatment of depression again is supported by these findings.

Rumination, often seen in the depressed, e.g., excessive guilt, self-blame, low self-esteem, can overlap with cognitive distortions as well as with OCD. In fact, depression can be viewed as a patient's inability to let go of focusing mainly on the negatives. They ruminate mainly on negative life events. The adjunct use of antidepressant-antipsychotic medications has been shown to be useful in treatment resistant OCD (Mohr, N. et al., 2002). Therefore, this medication combination targets another depressive symptom, and substantiates its use for depression and for decreasing the risk of suicide. This is another example of how targeting the extended depressive symptoms, and not just the mood, can increase the effectiveness of the treatment of depression. This is also an example for the “pseudo-placebo” effect.

Social withdrawal, i.e., lack of social support, has also been mentioned as a risk factor for suicide. Social withdrawal is found in almost half of suicides. It is known that atypical neuroleptics (atypical antipsychotics) improve “negative symptoms,” including social withdrawal, at least in psychotic patients. Although this cannot be extrapolated to depression, further studies for the use of adjunct atypical antipsychotics or “dopamine system stabilizers” in clinical, non-treatment-resistant depression is supported in the context of the present invention. Withdrawal is another symptom that can be targeted in the treatment of depression.

Additionally, it is known that atypical neuroleptics have a positive effect on improving depression in psychotic (schizophrenic) patients. atypical neuroleptics also reduce hostility and the risk of suicide in this patient population. In a study lasting one year in psychotic patients, the annual suicide attempt rate with atypical antipsychotics showed a 2.3 fold reduction compared to patients receiving haloperidol, an older antipsychotic (Glazer, W. M. 1998). While these results cannot be extrapolated to MDD, in light of the arguments presented herein, further studies are warranted in this regard in depressed patients.

It is also known that suicidal individuals often find their thoughts restricted to a narrow range of topics and that they tend to restrain their options prematurely. In other words, they display cognitive impairment and cognitive distortions. Again, the atypical antipsychotic medications have been found to have a beneficial effect on cognitive impairment, at least in psychotic patients, as measured by psychological testing. Although this also cannot be extrapolated to MDD, it provides additional support for the adjunctive use of antipsychotic medications with antidepressants in unipolar, non-treatment-resistant depression. These symptoms from the extended list may also be targeted with medication (where again the “pseudo-placebo” effect is utilized).

Some reports have identified a so-called suicidal depressive syndrome, which is not listed under the DSM-IV, in which patients with major depression are at a higher risk for suicide and also have feelings of worthlessness, anxiety, depressive delusions and more sleep disturbances. Anxiety itself is a unique and short-term risk factor for suicide, and in patients with major depression, anxiety predicted 93% of suicide within one year of assessment. Indeed, patients at highest risk for suicide are those with more severe anxiety combined with depression. Therefore, the addition of a neuroleptic, with its anxiolytic properties, would be justified. This points out the benefit of targeting the “extended” symptom list of depression one by one, as provided in the present invention.

In an article reporting on the treatment of resistant major depression with olanzapine and fluoxetine, the authors did not find a statistically significant improvement in depression with the Hamilton rating scale for depression, but they did find it with the Montgomery-Asberg depression rating scale (Shelton, C. R., et al. 2001 (a)). Nevertheless, the authors did find the improvement of depression clinically significant. What appears to have gone unrecognized by these authors (and others in the process of replicating the study) is that there is a specific, significant difference between these two psychological rating scales. Namely, the Montgomery-Asberg depression rating scale puts a relatively higher emphasis on anxiety (1 in 10), while the Hamilton rating scale for depression rates psychic anxiety on a scale of 1 in 20. (Somatic symptoms/anxiety are measured separately) Additionally, the Montgomery-Asberg depression rating scale allows a 0 to 6 measurement of inner tension, potentially allowing more emphasis in the statistical analysis. In comparison, in the Hamilton depression rating scale, there is a 0-2, 0-3, and, for anxiety, a 0-4 scoring. (For replicative studies, it is important not to use a “simplified” Hamilton rating scale, where there is only a checkmark for the depressive symptoms, not allowing for any severity rating: This would make the Hamilton scale even more ‘insensitive’ to changes in anxiety).

Nevertheless, what the authors might actually be measuring (in coming up with a statistical difference in one scale, but not the other), is the relative improvement in anxiety. It is known that antipsychotics reduce anxiety. (Although this group of medications had been named “major tranquilizers” early on, it was because of the strange quietness or blandness (ataraxia) that the patients were displaying). Undoubtedly, other factors may also play a role in why the combination of antidepressants with atypical antipsychotic medication results in an improvement in treatment-resistant depression. We have already discussed above some of the key factors that can contribute to the improvement of depression by adding an atypical neuroleptic, or a “dopamine system stabilizer”.

It has been previously discussed above that atypical antipsychotic medications, at least in psychotic patients, have a beneficial effect on negative symptoms. Negative symptoms include the following: affect blunting, which may correlate to such symptoms in depression as decreased interest, concentration, and psychomotor retardation. The term anergia correlates with the symptom of decreased energy. Alogia, if due to depression, may be a result of decreased interest, psychomotor retardation, or decreased energy. Social withdrawal may occur for many reasons, but decreased interest, concentration, psychomotor retardation, guilt, hopelessness and cognitive distortions (which are all symptoms of depression), can also play a role. Even though these results cannot be extrapolated to MDD, it should provides support for the notion that atypical neuroleptics, as adjunct to the antidepressants, can be beneficial for the treatment of depression, and may target numerous depressive symptoms and have a “pseudo-placebo” effect.

In support of the novel approach of the present invention, in which different medications (and/or medication combinations) systematically target specific depressive symptoms for more rapid and/or more effective treatment of depression, a clinical observation is presented. When the treatment of certain psychiatric disorders, e.g. anxiety disorder, OCD, PTSD, is successful, the frequently associated (comorbid) depressive symptoms also are lifted. This substantiates the importance of the novel approach of the present invention that the more symptoms that are addressed and corrected “right away,” the better the chances for patient satisfaction and global improvement.

It is not polypharmacy to advocate a more vigorous treatment of depression, which improves patient satisfaction; provides a more rapid result in lifting depressive feelings and hopelessness, as well as anger and resentments that accompany depression, all of which would contribute to reducing the risk of suicide. More than one medication can be responsibly prescribed. As in all treatments, the final decision is always up to the patient and the treating clinician. Offering patients more than one option that includes the combination use of psychotropic medications can have many advantages. In this way, we are involving them in the decision-making, along with presenting the risks/benefits, side effects of the medications, and available alternatives.

Thus, the present invention places more weight on the use of medication combination, preferably by adding an atypical neuroleptic or a “dopamine system stabilizer.” In psychiatry, one is not afraid to prescribe more than one medication to patients, and the treatment of depression is not an exception.

The treatment of depression can be started immediately with more than just one medication, such as an antidepressant. For insomnia, a sleeping pill can be prescribed temporarily, such as zolpidem. The combination of an SSRI and an atypical antipsychotic, or a “dopamine system stabilizer,” can provide great advantages, such as the prevention suicide. Thus, by treating depression vigorously, one can prevent suicide. There is another benefit to aggressively treating depression, namely, the reduction of medico-legal liability by preventing suicide. Unfortunately, there has been an increasing tendency over the past decades to blame someone when a patient commits suicide. One in six patients with major depression seen by a psychiatrist commits suicide. About 15% to 20% of all patients with serious affective disorder will commit suicide. One study by the National Institute of Mental Health revealed how difficult it is to accept the loss of a loved one, and almost none of the mothers of leukemic children are able to accept the absence of human causation in their children's death.

In the current health-care environment, clinicians have to adjust to too many variables. The standard of care also is changing, which at times creates a huge challenge to clinicians. This is especially true in Managed Care settings. There are publications that address the challenges that clinicians face, but unfortunately this does not reduce the health-care providers' medico-legal liability. Predicting which patients will commit suicide is an impossible task, and there are no models of suicide risk assessment that have been empirically tested for reliability and validity. It often is difficult for clinicians to assess the risk of suicide systematically, due to the large volume of patients, and to limited time and resources. Information that is available often is limited. There is a continuum in the risk assessment, going from asking the patients if they are suicidal to performing formal systematic suicide risk assessment. It is known that the former technique is much more common. Unfortunately, the “no harm contract” is unreliable, and short hospital length of stay, rapid patient turnover, brief outpatient and partial hospitalization visits, and the split treatment in managed care setting, results in difficulties such that the suicide risk factors usually are not recognized by clinicians. Only the sickest patients are admitted to inpatient psychiatric units, and the average length of stay for depression/mood disorders can be as little as six days. The length of stay for patients whose medication needs to be adjusted may actually be even less, as patients requiring ECT decreases the average for hospital days. The assessment of suicide is further complicated by the fact that approximately 25% of patients at suicide risk do not admit to being suicidal. (However, in most cases they did communicate suicidal ideation or intent to family members.) Patients who deny suicide risk usually do not meet Managed Care criteria for hospitalization. In the best case scenario, when it takes 2 to 4 weeks for antidepressants to start working and to produce visible signs of benefits, it is a real burden for both patients and clinicians to manage inpatient treatment under one week! Yet, as the statistics reveal, currently this is the current situation. Unfortunately. the difficulties in the current health care environment do not reduce the clinicians' medico-legal liability. (Case vignettes from the Mental Health Law News may further substantiate worries about malpractice law suits and the mental health/insurance crisis. Prosecutors may twist the facts and suggest that the psychiatrist “opted not to personally evaluate the patient at the middle of the night,” thus winning the case.

The above description demonstrates that research studies should carefully weigh the importance of the above information. For example, it is possible that one medication shows a superior effect to placebo in a study by targeting a symptom that traditionally is not included in the diagnostic criteria set of that particular disorder. However, not recognizing how a medication might work, and yet getting an FDA indication for monotherapy for that disorder, can come with a great risk of withholding a more effective combination treatment. One example is bipolar (manic depressive) disorder. Antipsychotic medications also show a value during the manic phase of the bipolar disorder, and the atypical antipsychotics recently have been approved by the FDA for monotherapy in this disorder. Yet, these studies might have not stressed in their design a sufficient reassurance that the desired effect in treating the bipolar disorder came from other than the antipsychotic effect per say; that is, that it came from a true “mood stabilizing effect” of these atypical antipsychotics. It is known that about two-thirds of patients with bipolar (manic-depressive) disorder have a history of at least one psychotic symptom. Bipolar patients who are psychotic during one episode of affective illness are highly likely to be psychotic during subsequent episodes. Therefore, the overall symptoms and the well being reported by the patients may be strictly because of the antipsychotic effect per se. This also may be true if the patients were not overtly psychotic. Antipsychotics also have a beneficial effect on agitation, irritability, anger, anxiety, sleep disturbance, cognitive distortion, and thought disorders, which may play a role in controlling racing thoughts, as discussed above. It would be tricky in study designs if one uses psychological scales in testing for improvement of symptoms for bipolar disorder, yet some of the symptoms were non-specific to mood, and overlapped with symptoms to which antipsychotics have an effect. Thus, it is very easy to come up with a study design where one gets a “positive” result, but not because of any direct effect of the medication on the mood. Disregarding this possibility, and approving a monotherapy, may run the risk of withholding mood stabilizers, such as lithium, valproic acid, etc., in some patients. Not striving for the most effective treatment also can carry the risk of suicide in serious mental disorders. Clinicians, as well as drug companies, have a responsibility of not only weighing the risk to the individual, but also the risk to group as well. This is because one does not know who in the group would be affected. Therefore, for the management of the most serious symptoms, such as suicide risk, the most effective treatment should be used.

Therefore, it is important to design studies in taking the above into consideration. Different study designs also should compare the result of the antipsychotic and mood stabilizer monotherapies with combination treatment (because each therapy may target different symptoms). It also is important to use more sensitive testing methods, with specific attention to the “extended symptom list” of the particular mental disorder, therefore differentiating between the symptoms targeted specifically by the two different classes of medications. It has been noted that some of the symptoms may not be present in depression (or in bipolar disorder) in all of the individuals, yet if a particular medication only acts on that symptom the group as a whole may respond, separating the medication from placebo, but some patients not having that symptom may not respond. Therefore, that medication, if used in monotherapy, may withhold treatment from that sub-group. Due to the complexities and “pseudo-placebo effects” that have been described, it is important to compare new monotherapies (like the atypical antipsychotics), to combination treatment. In addition, new medications (in monotherapy) should be tested for their effectiveness in maintenance and prevention. There also is a need for the same considerations when investigating different agents for depression. Thus, scrutiny of the above described considerations for future studies, including those to be sent for new FDA indication/approval or for suggesting off label use, is therefore essential.

Therefore, by specifically targeting the various depressive symptoms with vigorous adjunct treatment modalities, and paying attention to the more extensive symptom list, the methods of the present. invention can change the standard of treatment regarding depression (MDD) and the prevention and treatment of suicide, which would result in different treatment decisions.

It was shown above of how correcting overlooked misconceptions in DSM can affect improved diagnosis and testing for depression and consequently in below of how better chances of patient satisfaction and global improvement can be achieve by targeting these various depressive symptoms very specifically or with adjunct therapies.

Further below yet another method of increasing patient satisfaction and global improvement through providing novel educational and/or marketing techniques is presented.

It would be worthwhile to distinguish the term neuroplasticity (an adaptation of the brain/or nerves to changes), or synaptic plasticity (changes observed at molecular or subcellular level) from “clinical neuroplasticity”, a term as used herein. In defining difference for “clinical neuroplasticity” from synaptic or cellular level: we propose and define that in “clinical neuroplasticity” correlating changes in sensations/motor functions/emotions can be mapped in the brain (not just simply saying that there is a “neurogenerative” process or new neurogenesis observed; and these changes in the brain should also be linked to a well explained functional neuroanatomy, and therefore allow to explain this phenomena to the average clinician, and or the public so that it would be easy to understand.

From this definition; saying that there is a change in the hippocampus in the depressed, but not being able of explaining how that change correlates (functionally, and specifically) to depression, or how the hippocampus plays a role in depression and mood; or to give simple factual correlation to findings at subcellular level (e.g. explanation that different antidepressant or drug effect(s) would result in change in some neurotransmitter receptors or changes in the subcellular level) would not satisfy the “clinical neuroplasticity” phenomenon. The description in the text (below)—with clinical orientation—would be an example to the description of the “clinical neuroplasticity”. These stories can also be well visualized/described.

We present a method to describe clinical neuroplasticity in depression not at cellular but at a clinical level. This description—in contrast to the neurotransmitter deficit/imbalance explanation as the sole (or main) reason for depression—can increase patient compliance with medication, decrease the bias or prejudice in the public against mental illness, decrease the patients' resistance and inappropriate use of less effective treatment or no medication treatment because of the existing misperception (and hostility against biological psychiatry). Therefore, this method of description can decrease the percentage of treatment resistant depression. We also suggest using the term synaptic plasticity describing changes at the cellular level and use the term clinical neuronal plasticity to describe changes at the clinical level. In particular this description can be used as a metaphor (in analogy to the neuroplasticity seen in stroke victims as the “turning of dominos”, “using [invisible] mitts” or “practice makes a master”). This can be described vividly, in easily visualized form(s):

(a), Although some may use the terms synaptic plasticity or neuroplasticity as synonymous, it may be better to separate the two phenomena. Synaptic plasticity as it relates to depression (and learning), is primarily referring to changes in the cellular, synaptic and molecular levels, with the focus on glutamate neurotransmission and NMDA receptors. One of the primary interests is on the volume loss of the hippocampus, with possible neuron loss during depression. It had been questioned if stress and elevated glucocorticoid levels (through oxygen radicals and “programmed cell death” [apoptosis]) may cause hippocampal neuron loss associated with subtypes of chronic depression. (Lee, A. L. et al, 2002; Duman, R. S. et al. 1999,). There is evidence that stress will cause a regression of dendritic process in hippocampal neurons producing loss of neuronal volume, this however, has been shown to be reversible with the cessation of stress. (Lee, A. L. et al, 2002,).

The synaptic plasticity model of depression also overlaps with the theory on the failure of neurogenesis (lack of brain cell growth) linked to depression. (Vogel, G. 2000, Malberg J. E. 2004). Neuroimaging techniques show smaller hippocampi in depressed patients, and antidepressant drugs and electroconvulsive therapy (in animals) show significantly more newly divided cells in the hippocampus. This is an addition to the recent discovery that had shown that the brain keeps producing new neurons into adulthood. (Vogel, G. 2000, Duman, R. S., et al 2000,).

(b), Looking beyond the changes in the hippocampus and receptor level in depressed patients, it would be worthwhile to separate the term synaptic plasticity from neuronal plasticity. In other words we suggest to use the term synaptic plasticity describing changes at the cellular level and use the term “clinical neuronal plasticity” to describe changes at the clinical level.

(c), In order to present a method to describe neuroplasticity in depression not at cellular but at a clinical level let us first explain neuronal plasticity, the capacity of the brain to respond to changes. This phenomenon had been extensively studied in some other conditions where the cortical representations of somatic perceptions can be mapped. (Spitzer, M. 1999,) (As the brain has no sense of pain, neurosurgeons could operate on patients while they were conscious [in local anesthesia or “woken up” after their sculls were opened] for example to remove a tumor, but to preserve brain areas that are essential to speech, vision or movement. During such operations it was discovered that part of the cortex that is responsible for processing touch sensations and is representing the different areas of the body, has a map-like structure, called “homunculus” in the cortex. Not only touching, but all senses are represented in topographical cortical maps. [See also: Spitzer, M. 1999,]).

(d), What the most intriguing is, that these cortical maps or cortical representations are not fixed, but have the ability to change if the input is changing (i.e. to show neuroplasticity). In a congenital malformation called syndactyly, the fingers are attached to each other (like in a fetal webbing). After the fingers are surgically separated, the borders between their cortical representations emerge in one week. (Mogliner et al. as referenced in Spitzer, M. 1999,). The opposite was also shown in animal experiments sawing the fingers together. Changes in cortical representation do follow this procedure.

In a different experiment (seen at PBS), a human volunteer was blindfolded for about two weeks, and it was found through a non- or minimally invasive procedure, that other brain areas started to “took over” the now unused visual cortex, and the cortical representations of the fingertips (touching) had increased.

It is interesting to compare that while it takes weeks for the antidepressants to start working, it also took week(s) to see neuroplasticity changes in the above experiments.

For a more complex adaptation neuronal changes may take even a year (e.g. cochlea implant). (Spitzer, M. 1999,).

(e), Similar cortical changes to the above animal experiments had been find in humans. It had been shown, that experienced violinists had a larger cortical representation of their fingers in their left hand compared to non-musicians as measured by magnetoencephalographic recordings. (Schlang et al referenced in Eisenberg L. TEN 2000, 2(4) 47-52).

(f), Another example of the brain's ability to respond to environmental changes was found with magnetic source imaging. Blind Braille readers who read with three fingers had substantial enlargement of their topographical hand representation in the postcentral gyrus compared to one finger Braille readers and sighted non-Braille reading subjects (Sterr et al. referenced in Eisenberg L. TEN 2000, 2(4) 47-52).

The above experiments are looking beyond the changes in the hippocampus (and are not constricted to the receptor level). Different emotions, or the changes in depressive disorders are not limited to the hippocampus, and other brain areas are also involved.

(g), Beyond the cellular changes in the hippocampus, and beyond the explanation of changes at intracellular level, the only strong support for the neuronal plasticity of depression, that we have seen published was the argument that the therapeutic action of antidepressants requires weeks, even though these medications block the reuptake or metabolism of norepinephrine (NE) and serotonin (5-HT) much more rapidly. The conclusion was that therefore the treatment of depression involves adaptation or plasticity of neural systems. (Duman, R. S. et al, 1999,).

(h), Yet it would be interesting to see a synthesis of clinical findings, supporting the neuronal plasticity model of depression from the clinical standpoint. We will present our viewpoint below; that will bring the psychological and biological explanations together, and will provide further understanding of depression. [These were never presented in this context before by other writers].

(i), In order to explain depression in the context of neuroplasticity, first we'd like to start with the “practice makes a master” metaphor or “turning of dominos metaphor”.

Although traditionally it was believed, that if stroke victims did not regain the function of their arm within a few months, then it was little hope for recovery, we know it now that this is no longer true. Supported by clinical data and not just animal experiments, we know that persons with stroke “learn” of not to try using their paralyzed arms or legs, and as times goes on this becomes an increasingly powerful conditioned response. However, by placing a restraint (a large stuffed mitt) on the patient's fuctioning arm, he/she is forced to overcome the tendency of not using his/her weaker arm. With physical therapy they are coached 6 hours a day to practice and improve the movements of their weak extremity. They are given tasks like turning dominos over (and cheered for their success). With practice and repetition comes a dramatic change within a few weeks. This phenomenon had been explained as a result of “an increased recruitment of neurons surrounding the area of the primary damage caused by a stroke”. The neurons that haven‘t been killed by the stroke, but are in the vicinity of the damage are sending out connections with other neurons. (Restak, R. M., 2001 and corresponding PBS video). This is the neuronal plasticity that we have also seen in other examples above. In principles we see a similar phenomenon when children's good eye is covered to force the weaker eye to “learn to see”. With practice we are relying on neuronal plasticity in a therapeutic way.

(j), Now, if this true on other areas, why wouldn't it be true for depression, or for the treatment of depression? Without our conclusions and without making a connection, we have published but not publicized data available to support, that most likely the same is true for depression. (Unfortunately these studies had not linked their findings to neuronal plasticity). Depressed people tend to focus on the negatives, and tend to ignore seeing the positives. Cognitive therapy teaches us to do similar repetitions, that is, to catch ourselves to have (negative) automatic thoughts, and make necessary corrections by doing an analysis of the facts on both the negative and the positive side. This is the practice that is similar to the “repetitions of the movements—turning the dominos” seen in stroke victims above. We just do not have a good visible “mitt” that would force us doing this practice. However medications do help exactly in that direction: We have mentioned above, that the problem in depression is rumination, the repetition and overt focus on the negatives, with cognitive distortion. Actually SSRIs used for treating depression are also working to reduce OCD symptoms, “the rumination”. (See also article about depression and rumination: Lyness J. M. et al., (1997). We have suggested that neuroleptics may also be helpful in many ways (including for rumination, as an adjunct to SSRIs), and are expected to help decreasing cognitive distortions, that are so characteristic of and are contributing to the depression.

(k), However, let see some experiments from decades ago that can be applied in this context, so that with our current knowledge they would clinically support the clinical neuroplasticity model of depression.

Although one of the experiments, (Haney, C., et al. 1973, also referenced in Yardley, K. M. (1982 b), and see also as related reference: Yardley, K. M. (1982 a),), was not designed to do anything with depression, yet has a great relevance to it. It shows the importance of how detrimental a ‘negative practice’ can be, even in “as-if” (or role play) situations. In a Stanford experiment they recruited normal healthy volunteers who agreed to take part of a “prison simulation experiment” for up to two weeks. They randomly assigned them to be either “prisoners” or “guards”. Unlike the guards, who had some minimal warm-up to the as-if event, ‘the prisoners were covertly inducted, without their conscious cooperation. For the sake of “realism”, they were arrested in the early morning, on false burglary charges, by actual members of the city police who were cooperating with the experimenters. The prisoners were then subjected to police interrogation and taken blindfolded to the simulated prison.’ (Haney, C., et al. 1973, also referenced in Yardley, K. M. (1982 b),). The “prisoners” were further subjected to humiliating and frustrating experiences (and their queries to the police if this had to do anything with the experiment were ignored). After a week the experiment needed to be prematurely terminated, “due to the ensuing emotional disturbances amongst the participants, particularly amongst the prisoners”. (Yardley, K. M. (1982 b),). The “prisoners” were feeling powerless, loss of control to the point of oppression, frustration, ‘emasculation’, anonymity, and arbitrary rule. The later in this case is really resulted in “learned helplessness” that we know as an important causative factor in the development of depression. While this experiment from the early 1970's looks cruel, and we can all hope that this kind of “experiments” can no longer be done today, they show the harmful effect of artificially being deprived of positive thoughts and emotions. This negative practice of focusing on the negatives, and to be forced to focus on the negatives, even in an “as-if” experiment, would result in an unwanted emotional disturbance. This is exactly the opposite of what we therapists and health care professionals want to achieve, and an example that “neuroplasticity” works both ways. In a commentary on the above ‘experiment’ Yardley notes that the outcome would have been different if the participants would have been brought out of the as-if situation every few hours or so to remind them of the as-if framing. (Yardley, K. M. (1982 b),). That means of shifting the balance between the negatives and positives. This is what depression therapy is all about when we give the patients the tools of doing this.

(l), In another experiment unemployed actors were recruited for a depression study. They were paid volunteers, and were asked to act and think as-if depressed, to walk slowly with a bent posture, and think that they are no good, etc. In two weeks they have shown biochemical and other signs of depression, and the actors reported that they had difficulty snapping out of the depression after the experiment was over.

All of the above supports not only the “clinical neuroplasticity model of depression”, but also the importance of practice to overcome depression. In this context this is very similar to the therapy of the stroke victims mentioned above. This “domino metaphor” (or “practice makes a master” and “using the (invisible) mitts” metaphor) can also be used clinically to motivate and educate patients about depression, and depression treatment.

(m), In a PBS film (“1940's House”), where “volunteers”—a family, lived “as if living in 1940's war time London” in historical cloths, with old fashioned appliances, with stimulated air raids, and mandated restrictions on their food supply (“as if there weren't enough”), the adult volunteer (“the mother”) who stayed in this “experiment” reported depressed feelings. When she could volunteer outside of this role-play in contemporary peace time nursing home, she reported her depression being lifted. This too shows a similarity to the above two “as-if” experiments, opposite of what we therapists want to achieve, and is another example that “neuroplasticity” works both ways.

(n), Interestingly, depressive symptoms also occurred in the “as if—24 hrs a day role-play, lasting for months” in the PBS' documentary the “Frontier House” (taking place under the re-enacted times and harsh condition of the American Frontiers”) where in one family, the mother expressed feeling depressed, and the father showed somatic concerns [frequently find in depression]—in this case about his weight loss. Interestingly, this was the family that “broke the agreed upon rules” of the role play, and made contact with the contemporary American society. Consequently they felt better in the later part of the “show” (i.e. their “as-if” experiment).

(o), The above (especially the first two example)—supporting the clinical neuronal plasticity model of depression—can also give an insight to the course of depression, and to the ‘natural’ tendency to relapse. It also shows of why is that so easy to relapse, if one stops taking the medication(s), or stops the ‘positive “domino” practice’. It was shown, that practicing cognitive therapy can be protective of the depressive relapse, and this is supportive of this view. It was also shown that the combination of antidepressant and cognitive therapy is superior to either treatment alone. [see Thase].

(p), One of the reasons for why the neuroplasticity model of depression is still lagging behind the other observations on the brain's power to adapt is that our technology did not allow us to “map” the cortical representations and changes that occur with the depression. Our brain imaging techniques are improving (George, M. S. 1994, Ketter, T. A., et al. 1994, George, M. S., et al. 1994, Rubin, E., et al. 1994,), but there is another way to assess and “map” changes in the brain.

The cortical representation of one's “inner world” may be also reflected by one's vocabulary. It had been shown, that in children, at an early age, words referring to the imaginary world (like fairies, dragons, etc.) shows a relative high ratio to reality based words in comparison to adulthood. (Deme, L. personal communication, Deme, L. 1975,). This “mapping” of children's vocabulary is in turn is also correlates with the finding that children has a greater involvement in fantasy, and have a higher hypnotic susceptibility. (Migaly, P. 1991).

Although it is not the same, but assessing patients depression (or feelings) with a psychological test (a word list of synonyms expressing different degree of depression), can serve as a “mapping” tool. With an analogy it is like the vocabulary in the RAM or the hardware of speech recognition software. The words used (thoughts ruminated) more often are stored up front (RAM), but other words are still recognized that are stored in the hardware. So mapping of the neuroplasticity changes occurring during or in the recovery of depression is also possible with a psychological tool relying on the vocabulary. (One has to be careful though to balance the testing with counseling and of not to alter with too frequent testing the “positive—domino—practice” encountered in therapy, or by the positive effect of the medications). [See and compare to M. H. Erickson's interspersal technique.]

(q), In helping someone to come out of depression (and one's inner world of focusing on the negatives), it had been shown that physical exercise has a value, and an antidepressant effect. (Russo-Neustadt, A., et al 1999, Blumenthal, J. A., et al 1999,). We also know, that in chronic pain, that frequently also overlaps with depression, physical activity has a beneficial effect. Moreover, physical exercise was also shown to be of value in connection with learning and neuronal plasticity. These similarities are intriguing.

(If indeed exercise is having a neuroprotective effect that can be shown by imaging techniques (Colcombe S J, et al 2003) than this would bring up the question that whether the decreased volume changes seen in the depressed is the consequence (in part) of the psychomotor retardation “decreased exercise”? Is it possible that just like with the serotonin (neurotransmitter) change seen in the “as if experiments” these volumetric changes in the brain could not necessarily be a causative, but an accompanying factor that could occur in anyone exposed to adverse environmental changes?—Or would these findings—i.e. from the depressive symptoms of psychomotor retardation isolation, (like decrease in exercise) just contributory to the hippocampal changes seen in depression? It is also possible that the hippocampal volumetric and/or morphologic changes seen in depression is rather than being an effect from the change of mood per se, actually may be a result coming from the process of learning and/or memory, since learning is involved in the process of mental depression (learned helplessness model); and/or memory/cognitive deficits are in the (extended) list of depression. All this new way of seeing the reasons for hippocampal changes in depression would fit in well with the findings that the hippocampus is involved in learning and memory, in which 5-HT and other neurotransmitters (NE, or NMDA receptor mediated responses) play a role. This recognition may also guide pre-clinical research for new antidepressants. (Focus may shift to the analysis of hippocampus and pre-frontal cortex in pre-clinical studies from the currently used animal models of depression). (Harvey, J. A. 2003, Meneses A. 2003, Coull, J. T. et al 1999, Rosenzweig, E. S. et al 2003).

(r), It had been questioned before that if for the depressed patients everything would go exactly their way for a few solid weeks, without disappointments, rejections or criticism while everybody would love them, would their depression go away? (O'Connor, R. 2001 p23). Well, it depends. These circumstances could definitely make everybody's life easier, but recovery to a large extent depends on “the domino metaphor” or practice mentioned above. (However, the “optimal circumstances” raised in the above question are so important that in our upcoming book we are paying attention to on how to achieve the most and get a harmony, a ‘full life’ not just recovery from depression.)

In fact the closing remarks in a book where there is a lot of discussion about neuronal plasticity emphasizes that we all should “watch our mental diet”. (Spitzer, M. 1999,). This means that we should watch the input we receive (e.g. through violent movies, discouraging news from within the society).

(s), There are arguments for the genetic transmission of susceptibility or depression. However, the increase in the rate of depression in the past decades cannot be explained by a genetic model. There is also data on stress and environmental events precipitating depression. (Putting less emphasis on genetics and more on environmental variables (i.e. that there are things we can change) in patient education would also reduce the hopelessness, (the lack of control). In contrast to the “neurotransmitter deficit” explanation, this would less likely to generate the sense “that there is nothing that I can do about my genetic makeup, therefore about my depression”. Therefore teaching the clinical neuroplasticity model of depression (above) [the need for the invisible mitt=meds, (and/or catching cognitive distortions/ruminated thoughts), and the need for practicing (positive/corrected thinking=cognitive therapy], can be more helpful and increase the patients cooperation and therefore compliance. (This is in contrast to the neurotransmitter deficit/imbalance explanation as the sole (or main) reason for depression—e.g. see Zoloft's TV advertisement and patient education material). The neurotransmitter deficit (imbalance) would occur in (basically) everybody in reaction to a strong (negative) environmental effect (as seen in the above “as-if experiments”). Therefore teaching the clinical neuroplasticity model of depression would also take away the “blaming, and self-blaming”. Consequently it would take away the desperate efforts in some, to fight the ‘acceptance’ of a mental illness (depression) with its consequent refusal for medication. (With this we could overcome one of the reasons why depressed patients do not seek treatment, why they discontinue their medications early, and why they are looking desperately for natural and herbal remedies instead of accepted and tested effective treatments.) It would also take off the edge for the debate for “medication”—no-medication dilemma (i.e. the struggle for fighting the disease without medication, the denial of mental illness, and the roots for its stigrna).

(The above teaching model would not negate differences in the sensitivity to depression, nor would it negate the roles of different learned cooping styles.)

In addition to the above what is the most intriguing is, that the environment does affect the expression of specific genes, at least as it had been find in rodent pups. Maternal touch, licking, (or touch with a paintbrush) affected gene expression and lead to receptor changes compared to the deprived control group. (See E. Rossi).

In summary for this section in looking the global picture, that is the role of neuronal plasticity in depression, the psychological and biological explanations indeed do blend together.

In this part we will explore yet another method of increasing patient satisfaction and global improvement through providing novel educational and/or marketing techniques: that eliminate or reduce prejudice against mental illness/depression, improve medication/and or treatment adherence, and therefore decrease the number of untreated depression. The same method can also be successfully used in other conditions where depressive symptoms are often present as coexisting condition (like in nicotine addiction/smoking cessation, overweight/weight control, pain management, addictions).

As we mentioned before: In targeting and specifically designing an approach of trying to solve (most/all) of the patient's problems—either pharmacologically and/or with psychotherapy can result in greater success than separate individual strategies alone.

Therapists and parents (unconsciously) know that understanding, unconditional acceptance, and limit setting are some of the most important factors and tools that allow an impact on others. However it is also true, that we can only change ourselves and (directly) no one else. To facilitate a change in others, we can do much better, if we are willing to revise what we can offer to the other person. The same principles apply to patients coming to medication clinics or to psychotherapy.

We put the emphasis is on how the health care provider should revise his/her own approach, and maximize of what he/she can offer to the clients, in order to come up with a combination of viable alternatives, (in discussing risks/benefits) that is appealing to the clients. Hesitancy and non-compliance can be drastically decreased in this way. This is true for any treatment or helping approach. We feel that way too often we “blame” resistance on the clients (and we do not mean pharmacological resistance, but their compliance. However, decreased compliance plays about 20% role in pharmacological resistance. [Thase, 2002 (b)]. When the clients' needs are met, there is nothing that they want to object to, and that is a winning approach. Let us elaborate of what we mean on this bellow, giving an example. We have adapted some parts from our own manuscript to exemplify the problem and principles to its solution:

“We health care professionals do not pay much attention to the hierarchy of human needs and on how to manage multiple problems in life all at once.

This is one of the main reasons for non-compliance, resistance, and in case of smoking, the high relapse and poor quitting rate among smokers.

Just imagine the following conversation when a doctor recommends his/her patient to stop smoking. What would you say to the patient's response if you would be the doctor?

“But doc my life is a mess right now! I just lost my job, there are constant arguments at home. My older daughter is not listening to me, she is running away for days at a time, and when she comes home, she doesn't respect curfew. She is acting out and getting into trouble. My smaller kid is failing at school. My spouse is a nervous wreck, always picking on me, and blames me for everything. When we don't fight, I'm getting the silent treatment. In top of all I also have the shared responsibility to help out an elderly relative with Alzheimer disease. And doc are you telling me to quit smoking now???”

Well what would you say to this? Would you become speechless? This is our point. There is a hierarchy in human needs and the lower things in this hierarchy needs to be attended first (that is those that have the highest priority). Clients need to take care of the most important aspects in their life to get a sense of harmony. [The same principles also apply in the treatment of depression].

The situation, the timing seems not right for quitting, and it doesn't seem important and emergent enough. The tools/skills our client has is also not matching up with the task of quitting and solving his/her problems at the same time.

There may be a wish for quitting but it is not going to be implemented to action . . . or . . . at least not until we can come up with brief and effective techniques, self-help materials that at this stage of reading [the manuscript of our planed] book really seems impossible.

So can we promise miracles when 50% of marriages fail in divorce, and 50% of those who remain in marriage reports unhappiness? . . . and we didn't even mention parenting, getting along with “that” difficult person at work, or other stressful situations.

People wish to change things first that are the most bothersome for them. Smoking is the number one preventable disease, but as so many smokers feel, quitting is not a high priority to them (as yet) . . . They just cannot imagine a fairly simple solution that would address all their problems. So myths about the “extreme difficulty” to quit smoking is going to remain very much a reality for them. Yet there is a solution!

We want to point out that we can do better then what we are doing now by paying attention to the hierarchy of human needs, by taking care of what comes first, and also by learning of how to manage multiple problems all at the same time. This is what our (planned) book is all about. The good news is that even if the problems are many folds, the same basic new skills that we acquire could be used in many areas of our lives.

When our clients needs are met, there is nothing that they want to object to!

Their resistance will melt away when their struggles about difficulties in life subside and they learn of how to meet their inner needs without the cigarettes” [or without hanging on to their current problem].

In (the manuscript of our planned) book this is what we emphasized:

“We have to go beyond simple techniques, or substitutes. Our book shares something new, a knowledge that will help you setting and achieving your goals, and transform your life to the better. When smokers go beyond occasional urges and start struggling with quitting it brings out of them an irritable, argumentative unhappy person that they do not want to be. This is a major setup for relapse. Instead of this we offer you to make the best out of this book, as it will bring the best out of you! What a different experience! . . . and you can make that happen! This is what this book is really all about, a lot more then just about stop smoking. It will help you to transform your life to the better. This book is also about health, harmony, and personal growth, of how to meet your inner needs. The success lies within that, your success!”

We had applied the following principles to our (planned) book:

(a), Instead of demanding a change from the clients, we changed and adapted to the clients' need. We said:

“We all know that it's not realistic, but it is still a human nature to wish for the others around us to change and make all the effort to adjust to our wants. In contrast, all (other) books seem to insist that it is the reader who should change and try something different. At times this doesn't seem fair, so you resist (and we all resist). Wouldn't that be nice if for once the author would change and adapt to the readers' needs? Wouldn't that be nice if the book would start with an expectation on he author and not with what the reader is supposed to do?

(b), We also made an effort to describe our new innovations and breakthrough by using simple language, and describing them “as if they already knew these”. We said:

“Can we describe to you not just traditional techniques to quit smoking, lose weight, deal with stress etc, but new information, breakthroughs, and still make you feel as if somehow you already knew that? If we can, and you can relate to it and accept it as “yours” that certainly would ease up the learning process. You want to quit smoking easily, and we too want to make your job simple. So let's see of how we can deal with this paradox and match this seemingly impossible requirement.”

(c), We needed to communicate not just at cognitive, but at emotional level. We said:

“Some songs get to the heart and it gives you a calm, uncomplicated feeling. Great novels certainly have similar effect, and grab your attention through evoking different emotions. With this you become far more then just a spectator and you engage in the story.

To help you quit smoking and achieve your goals we will share with you a great deal of information and at least in the cognitive therapy chapter, we would need to rely on reasoning and your logical thinking. However that's not what made you to smoke and (by itself) it won't make you to quit either! In order for our book to successfully help you, we would have to balance that information so that as a sum it affects your feelings. Not just any feelings, as scaring won't do! We would have to elicit positive feelings.”

(d), We were also very attentive that our clients (the readers) could have a negative frame of mind that needs validation. While we talk about positives they may feel the opposite, and be hesitant or very discouraged. So we also relied on universal human experiences that all people can relate to. We came to the conclusion that: “It seems that we are all striving to better ourselves, and contrary to the popular belief there is a great appreciation for change after all!”

(e), We needed to raise hope. We said:

“Hope is needed in everyday life and is also needed for success. What would your efforts to quit smoking be if you wouldn't have confidence about yourself, if you wouldn't trust and believe that you can succeed?

We can only expect you to change if you can see for yourself that there is a good chance for success.

Maybe one of the best ways to raise hope and increase self-confidence is to give you a new, direct personal experience that change is possible. They say after all, that “seeing is believing!”

Your decisional balance toward a smoke free life, will stand on a much firmer grounds if your mixed feelings about quitting are resolved first and your resistance and hesitance are removed. Actually, with the gentle approaches you will learn, that you may do much better if we do not blame you for psychological defenses (like denial and rationalization), since they can be viewed differently, where they become no longer to be an issue, and they just melt away.”

(f), We also stressed that all of us wanting to help people to quit smoking need to keep in mind that: “Change starts where you, the clients are, not where we think you need to be. Therefore we have to meet you here without any nagging.”

(g), We kept in mind that “You must feel understood, and we must be attentive to your needs and wants. You and other readers may show diversity in many things. Your priorities, readiness or hesitance to quit or your learning style may vary, but one thing is common to all, that would lead you to success. We need 1; to show you that you can do it, and 2; that it is going to worth it. Things that you value the most will support you in your decision in that.”

(h), One of the most important pointers for facilitating a change in clients and to help them succeed was in stressing (in the manuscript) that: “You have to come out with a gain (in your analysis) in order to be smoke free. No one can expect you to change unless your benefits from being smoke free exceed the benefits you perceive now from smoking. To assure an overwhelming success we (the author) have to adjust what we can offer to you, so that you definitely would come out with a gain. You need to meet complete satisfaction from your new lifestyle . . . but as we said before: When your needs are being met, there is nothing that you want to object to! . . . and that is a winning approach!”

(i), We were looking for ways to achieve change naturally: “You can see that the author changes himself, his approach and of what he has to offer, again and again for every bit of little change that we are going to expect from you. In this way change would come naturally from within yourself. That makes things so much easier! It's a common goal that we (you and me) share. When we all are on the same page and we focus our energy on reaching a better life, rather then fighting or resisting it along the way, things can speed up to the better, and give all of us an uplifting satisfied feeling not just success . . . ”

(j), “We will bring the best of the best techniques for stop smoking and for problem resolution for you. In doing that we may stumble upon some breakthroughs never published before, and present to you new ideas, or old ideas from new perspectives in innovative ways.

If all the so-called “revolutionary” programs out in the (book) market would live up to their promise and their names and fame, we wouldn't dare writing just another book. However, as we will show, the quit smoking rates can be improved quite a bit, and the quit smoking process can be made far easier.” (We also attended to communication and listening skills, finding solutions for relationship/marriage, parenting, and work issues, and addressing stress management, setting priorities, and problem solving.)

“Permanent change rarely comes in splits of seconds. All we can do is to explore new ways that gives shortcuts and new directions to success. This is what we aim with our book.

However, rapid change does exist and can occur. With rapid change and one can also side step relapses.

Nothing is totally effortless, you will still need to go through this book, but we hope that you will enjoy that journey!”

The above principles can be applied basically to any problems, not just in helping people to quit smoking, loose weight, find a better resolution for their depression, or to stick to their medication regimen. It can be used in therapy, healthcare, (better) education, marketing and other areas in life.

Appendix:

Psychological scales [psychological (psychometric) testing]: Principles for creating better psychological (psychometric) tests, or using combination of existing tests (or parts thereof).

As mentioned we can achieve a better result by targeting the resolution of each and all depressive symptoms. Therefore the improvement of these symptoms (and their synonyms!!!) should get a weighted emphasis in the psychological testing. [That includes the symptoms for the acronym “SIGECAPS”, but other symptoms (“ARS, AHIV, W, H/H, S, CD/CD, S, O/O, URSSI” or “AHIV AROWS CD/CD H/HORSS IS U”). should also be incorporated in testing for depression (See later bellow in this Appendix).

The development of such psychological testing scale could be more precise in assessing even subtle differences in the efficacy of antidepressants, the differences between antidepressant medications (e.g. now believed to be equally efficacious), or in assessing the differences between a particular antidepressant and a combination therapy. With such a scale (psychological testing instrument) a timeline (e.g. for more rapid/earlier) improvement, could be also better assessed. The differences in patient symptomatology could also better guide treatment (like emphasizing the need for choosing a combination treatment right away in treating depression). Furthermore, with a more sensitive test, the (currently fairly large) number of patients required for a particular (pharmacological) study can be reduced, therefore making it feasible and practical to conduct such studies advancing science.

It seems that with quick and brief scales we only get a rough estimate of depression. In combining the power of various (i.e. more) but not “equivalent” tests, we are likely to get a more sensitive assessment tool.

(On “not equivalent tests” we mean, that they were not designed to strictly measure depression only, or they were designed to measure other diagnosis than depression, or other symptoms not in the DSM criteria list.—On the other hand, under “equivalent scales” for depression test we mean tests that had been designed or usually used as testing for the diagnosis of depression [and depression only].) (On the negative side of using an extended list, it is true, that the timing needed for the assessment would increase, and depressed patients have shorter attention span. [Therefore if needed patients may need to take a brake during the test]. However, we need instruments to better guide everyday routine clinical treatment, as well as research, therefore leading to better practice guidelines.)

At times in the depression literature more than one tests were used. (See e.g. Shelton's paper). However using tests that targets the same symptoms would give pretty much the same result. So more (tests) are not necessarily better. The Beck depression inventory, the Hammilton, Zung, MADRS scales for example are basically equivalent. In fact depression tests had been validated to match the “gold-standard” Hammilton depression test.

What we suggest is very different: The testing should target not only the traditional depressive symptoms but also the ones that were left out from the diagnostic categories. Therefore we should adapt and integrate into the depression tests (whole or preferably parts of) the other existing tests that measure these other cluster of symptoms. This is regardless of that these tests were not necessarily designed to test depression per say. These tests might had been designed to test anxiety, OCD, stress, burnout, anger, impulsivity, personality disorders, schizophrenic symptoms or general quality of life etc.

In addition, we suggest that these “extended” symptoms should be routinely and systematically checked under both clinical and research conditions, and not limited to their sporadic or partial inclusion into the test. The power coming from using most of these extended symptoms is synergistic.

(However we are not interested in parts of these tests that check for symptoms that would (usually) not be part of the “extended” depressive symptomatology.) Just like in the first part, here too we have separated these various categories (shown with lettered [a,b,c] marks). These are meant only as examples and it should be understood for those skilled in the art that many other variations exist that should not limit the scope of this invention.

The benefit of combining these clusters of tests becomes significant when more than 2, 3 or 4 of these different test categories (or parts thereof) are applied. (One should be receptive also to the fact that the attention span and interest of depressed patients are decreased, so one should try to balance this new test to include items from all areas, yet to keep the length of the test to the minimum.) The scoring of the test may also be “weighted” in its “importance” that is the obtained scores from the different areas do not necessarily need to be “equivalent” in their power in assessing the severity and characteristic of the depression. This is best designed when it also shows how it affects the patient's overall quality of life. Measuring the change in the symptoms over time (lifting of symptoms) is also important. Another possibility for scoring the test is to rate (or rate the change of) each symptom separately and score the test (and the improvement of the patient) accordingly. This approach may separate the effectiveness of certain medications or medication combinations. Symptoms that have more weight in contributing to serious consequences (or those that decrease or take tools on the quality of life the most)—like anxiety, rumination/disturbing thoughts, impulsivity, anger, or suicidal thoughts—should be scored more heavily at least in one preferred application of our method.

(a), Since the antipsychotic medications have desirable effect on the negative symptoms of schizophrenia and psychotic disorders, and the negative symptoms [like: affect blunting, social withdrawal, anergia, alogia,] can be interpreted as being similar to some of the depressive symptoms without psychosis, therefore in assessing depression with psychological testing, such scales should be added and adapted in the testing of depression.

The Scale for the Assessment of Negative Symptoms (SANS), (Andreasen, N. C. 1982) and Positive and Negative Syndrome Scale (PANSS) (or parts of) with specific attention to their negative scale can be added or adapted to the depression rating. Although the normative data was developed and used for psychosis, the type of questions these scales investigate can show a promise in testing depression more accurately. For example SANS rates such items as “unchanging facial expression” or psychomotor retardation (under the name of “decreased spontaneous movements”). This scale also assesses a decrease in spontaneous gestures, affective non-responsibility, lack of vocal inflections (monotone speech), poverty of speech, and delayed latency to respond. Poor eye contact, anergia, decrease of recreational interest and activities, sexual activity, social inattentiveness are also rated. These items are all essential in assessing depression, and clinically with less sensitive questions we all measure them. On its negative scale PANSS also rates blunted affect, emotional withdrawal, lack of interpersonal empathy (poor rapport), social withdrawal, and reduction of normal flow of conversation associated with apathy. It also rates stereotyped thinking, the spontaneity-rigidity of thought content.

(b), In testing depression, cognitive distortions should also be assessed more elaborately (they overlap with impaired reality testing). [e.g. Cognitive Bias Questionnaire {from Krantz, S. et al. cit. A. Nezu et al. 2000.}, and Cognitive Triad Inventory {from Beckham, E. et al. cit. A. Nezu et al. 2000.}].

(c), Similarly, since there is a cognitive impairment in depression (Forster P., 1994.), it should be also measured. Either directly or indirectly, but some of the depressive symptoms affects cognitive functions. Lifting of depression improves cognitive function. Atypical antipsychotic medications have a positive effect on cognition in psychotic patients—and it can be measured. Although this cannot be extrapolated to depression, but assessing the cognitive improvement after the treatment of the adjunct antipsychotics in depressed should not be neglected, and may require further studies.

(d), In testing depression, the assessment of ruminations should also get (more) emphasis, as it overlaps with the obsessive compulsive symptoms. (use OCD scales and tests to assess rumination).

(e), Furthermore, it is known that in the assessment of pain, psychological scales have been developed, where the emotional tones of the words have been rated by the patients (to describe the characteristics, quality/intensity of pain). (See Melzack R, 1973). Therefore, in assessing depression, emphasis should be also made on developing and using such scales that assess the emotional tones of words that describe the symptoms of depression.

This can be done in two different ways (or by combination of them).

1, First the different adjectives, or emotional tones of the words can be rated to describe the characteristics and intensity of the emotional—in this case depressive feelings. (In the same token similar scales can be used also to better rate anxiety, rumination/obsessive-compulsive symptoms, or other emotions).

The following indented part gives an example of how much difference there is between the words (or cluster of words) selected by the patients. (Similar tests had been developed by Lubin, B., and by Lubin, B, and Zuckerman, M.)

There is a huge difference between these words describing depressed feelings:

unhappy, discouraged, feeling down, sad, depressed, sorrowful, gloomy, miserable, tormented,

Similarly there is a difference between these words describing non-depressed/normal feelings:

undaunted, undismayed, unsubdued,

so-so, fair,

fine, normal, back to usual,

comfortable,

good, well, harmonious, grate, splendid,

There are also subtle or not so subtle differences in describing happiness:

pleased, glad, happy, delighted,

cheerful, rejoicing,

high, euphoric, ecstatic,

If a patient would rate a similar (filly developed and standardized) scale selecting all the words that apply to his/her feelings either daily, weekly, that could give a more precise global picture on how the depression is changing, than the currently available scales. Of course, other measures such as the neurovegetative signs of depression, or the rating directions emphasized above are also equally important.

(f), In that new test for assessing depression the patterns for aggression/risk for suicide should also get more emphasis, since it may guide treatment choices. Analysis of MADRS suicide thought item (See Keck P. E. at al 2000 (b), 61, (along with other scales to assess suicidality).

(g), The assessment of anxiety, somatic symptoms and sleep disturbances should also be incorporated with an increased emphasis. [use scales to measure anxiety, and other symptoms (now reclassified as depressive symptoms)].

(h), Assessing impulsiveness, (e.g. with Barratt Impulsiveness Scale score) and anger/hostility/violence. (May use some parts of personality scales as well).

(i), Assess stress (e.g. Spilberger), and cooping/problem solving skills, and burnout questionnaires.

(j), Assess motivation.

(k), Assessing quality of life-scale

(l), Assessing basic beliefs about suicide, trying till succeeding or giving up, and about problem solving, problem analysis.

In addition ratings from observers description can also be used (see further down).

There are also a number of other tests that can be relied upon (see reference and above).

For clarity we'd like to emphasize that the combined use of existing psychological scales can be extremely beneficial.

The above were provided only as examples, and did not mean to limit the principles listed above.

Certain other questions may be added to the test(s) e.g. validation purposes.

It is important to stress that symptoms from the “extended” symptom list (in addition to depressed mood and “SIGECAPS”-“ARS, AHIV, W, H/H, S, CD/CD, S, O/O, URSSI” or AHIV AROWS CD/CD H/HORSS IS U) should be included in a systematic way, that is not just randomly selecting some for one test and other symptoms for another test. The intended increased sensitiveness to test/and monitor depression can be achieved through following this recommendation. Also, we would recommend that these extended symptoms to be checked also at routine clinical interviews and assessments, not only at clinical research or drug development.

As stated previously, correcting overlooked misconceptions in DSM can affect improved diagnosis and testing for depression. There is a better chance of achieving patient satisfaction and global improvement by targeting these various depressive symptoms very specifically or with adjunct therapies. This is—as above—how we define systematic application.

The following examples are intended to illustrate the invention and should not be construed as limiting the invention in any way.

### EXAMPLE 1

Psychometric Testing—Ouestions and Description of Innovative Scoring Guidelines

1. Introduction

The following depressive symptoms is weighted and scored more heavily, as the changes in these symptoms may be the most bothersome, may play a more important role in depression than other symptoms, and can be specifically targeted with medication combinations, i.e., pseudo-placebo effect, therefore resulting in a quicker and/or further resolution of other symptoms, such as hopelessness and helplessness, as well as alleviating the patient's problems, such as relationship issues, once anger impulsivity is resolved, and improving their general overall well being.

Symptoms: Suicide for its serious consequence, Sleep disturbance, Rumination/OCD, Hostility/anger/irritability/impulsivity, Energy, Stress/Traumatic Stress, Anxiety, Somatization; as well as to a lesser degree: Guilt, Cognitive distortions, Unjust, resentment,

The test is scored not according to the total number of items that are added together (raw score), but rather according to the scores one gets for each symptom cluster. The more important symptoms (that can be more readily targeted/and or indirectly act on the mood and/or the helplessness/hopelessness more “intensely,” and the ones that determine the patient's overall well being, that is the relative importance for the patient and clinician for relieving that depressive symptom, should determine the importance or the “weight” of that score. In the scoring, it is novel that those depressive symptoms scored more heavily are the most bothersome, and/or are those that through the pseudo-placebo effect can be modified play a more important role in the overall resolution of depression and hopelessness.

Some of the questions are overlapping, and therefore can be scored for more than one symptom. Patients may need to take a break if the test is long. Not all of the following questions need to be in the test, while other questions can be included. In addition to the above, other depressive symptoms also can be tested. In a preferred application while administering the test the questions would be mixed together, not separated by symptoms.

In scoring the proposed scales, one should subdivide questions to symptom clusters, as it can be informative on the need for further improvement in addition to the total scores. In addition, it should be noted that if the total score improves, but mood, or other subscales lag behind, then the medications in monotherapy may not be comparable to other antidepressants or medication combinations for exactly that reason. All or most of the subscales should show an improvement for optimal antidepressant effect. However, some subscales, such as interpersonal/relationship skills, should not be expected to show “normal” range from medications, as it takes learning and practice to perfect the improvement on these scales. The medication, or treatment methods, however, can clear the way for the individual to progress at such subscales by removing other stressors and depression. In scoring the test it is imperative to compare the scores to baseline, but also to realize that for optimal harmony and good relapse prevention, the scores on the interpersonal/harmony subscales can be improved beyond the baseline.

The scores can be further “fine tuned,” e.g., barely or none of the time: never=0, some of the time, or a little of the time: seldom=1, (occasionally=1b), often=2, (most of the time); almost always=3.

Questions also can be scored according to negative or positive effect or depending if the same question is asked in a positive or negative way, e.g., “I feel tens” vs. “I feel calm;” (−) reverse scored.

Questions also can be scored according to their having a protective or contributory effect on the symptom. In scoring the test certain protective questions [like creativity (−) scores, protective answers on stress, positive balance on feelings, or protective answers on cognitive distortions (catching and correcting automatic thoughts, analyze things factually)] may be used in a way to balance out some of the (non-life threatening) other scores in the depressive symptom list.

2. Examples of Questions

Suicide for its serious consequence:

I have short and long range goals in life (−)

Even if certain things do not work out for me, there are still abundant opportunities in life that waits for me. (−)

I had thought about dying or I wanted to die,

I thought the world would be better of without me, or I wish I was never born,

My thoughts are so unbearable I cannot take it any more,

I fantasize others feeling sorry for me, or having serious guilt about letting me die,

When I have hurt myself or told that I wanted to die, I just wanted others to pay attention to me, or to get help,

I have hurt myself before, or tried suicide,

I feel that people who want to die should be allowed to kill themselves,

Sleep disturbance:

I have problems sleeping at night,

By the time I go to bed it is late at night, yet I need to get up early in the morning (I do not allow, or my circumstances do not allow enough sleeping time for me),

I wake up during the night, and cannot fall back to sleep,

I use sleeping pills to help me sleep,

Rumination,/OCD:

Now that you had some orientation about cognitive therapy and automatic thoughts, how would you rate the following for the past week:

How much time do you estimate you have spent a day ruminating about negative life events, stressful events, or had angry/irritative or catastrophizing anxious thoughts, or thoughts of negative self-worth? (0 hrs/day, 0-1, 1-3, 3-8, 8+),

How much did this rumination interfere with your daily life (robbing your peace and harmony, your relationships, your job performance, your concentration, or interfering with your sleep and energy, and your self-esteem) (none, mild, definite but manageable, substantial impairment, incapacitating),

How much distress did you experience from this rumination (negative self-talk)? (none, little, moderate, but manageable, severe, near constant disabling,),

How much were you able to catch yourself and get a control over this rumination (negative self-talk)? (e.g. balancing out with positive thoughts, analyzing for reality and correcting your thoughts, dealing with your thoughts in any other way so it would not negatively affect you feelings-hashing the thoughts away, joking it off. to regain control)? (complete control, much control, some control, little control, no control)

I cannot get my mind off certain thoughts,

I often daydream or dream about bad things or revenge.

I have had experienced intrusive thoughts of times,

I catch myself tensing up or speeding when I have intrusive thoughts of being violated against,

I feel that I had been “infected” by the trouble of the world or the stress of decreasing morale,

Negative feelings intrude my life,

I have thought of wanting to harm others, or myself,

At times I feel like going crazy,

My thoughts are unbearable at times,

Most days lately, I have violent or repulsive images,

I have frequent thoughts that my body is disfigured or not perfect,

I have frequent thoughts that I may be ill, or that my body/organs are not functioning well,

Certain unimportant questions or topics keep coming to my mind that I cannot get rid of,

I think of seeking frequent medical attention to see if my body is working right,

I keep thinking to certain past situations in my life,

My thoughts are so unbearable I cannot take it any more,

Hostility, anger, irritability, impulsivity:

Others accuse me or tell me that I'm often aggressive, or disruptive,

Do you feel angry or snappy often, several times a day or a week?

Do you have anger outbursts monthly, weekly or daily? (circle). Does your anger or it's consequence bother you or others?

Dou you have regrets or guilt after being angry, or do you feel justified? (circle),

Do you take offense readily with anger, withdrawal, counter-offense, or revenge? (circle),

Are you at times violent to others or to yourself, or do you destroy property (including by punching, kicking, or throwing object)? (or having such a thought)? (circle)

Do you behave restlessly (e.g. ignoring traffic rules)?

I argue a lot,

I'm impulsive, acting without thinking,

I'm screaming in my anger,

I'm whining,

I am always looking for realistic alternatives rather than acting impulsively. (−) (exactly true, moderately true, barely true, not true at all)

How angry or annoyed do you feel in the following situations?: (Very Little, Little, Moderate Amount, Much, Very Much ) You buy something and discover that it doesn't work; The person you are talking to is not paying attention to you; When others blame you for their own mistake; When others tease you, mock you or make fin of you; When someone just turns in front of you than goes much slower than the speed limit.

I get mad without much reason,

How many hours a week or day do you listen/watch hard rock music (if it is often with violent lyrics)? Never, 1-2 hrs/wk 3-7 hrs/wk, 8-14 hrs/wk, 2-4 hrs/day, 4-8 hrs/day, more-even when I go to sleep,

I lose my temper easily, I can get angry or upset easily,

Other people usually like me, (−),

I never tell a lie, (validating question)

I always think before I do things, (−)

Energy:

I cannot keep up with the chores at home or at work (behind schedules, untidy, disorganized place/work, problem with self-care, neglecting grooming, dressing, eating),

I feel worn out, or have too little energy,

I feel burnt out,

I feel tired, having little energy,

Stress:

I had been fired or I'm afraid that I will—as I cannot keep up with the demands,

I'm quite capable to deal with unexpected events, (−)

I'm resourceful in solving problems, (−)

I think of problems as leading to a no-win situation. (always, often, rarely, never)

I cannot relax easily,

If I'm delayed, for any reason I get inpatient,

Once you get worked up or rushed, it is difficult to calm down,

Interruptions in my work upsets me,

I'm tense most of the day,

I have good peer support when I have stressful experiences, (−),

My life is satisfying,(−),

I feel connected to other people, (−)

I am a calm person, (−)

I keep a good balance between work, home, sleep and my free time, (−)

With little provocation I get easily angry or irritable,

I lack close friendships,

I lack an intimate relationship,

I wish I could avoid contact with some people at work,

I feel safe at work and home, (−)

When I was younger this was not the kind of self-fulfilling work I dreamed about,

I live my life according to my basic beliefs and principles, (−),

I usually find things in my life (at work, at home, or at my leisure time) where I can be creative, (−),

I like routines and repetitions, I have difficulty with change,

I accept that changing and adapting to situations is part of life,

I take changes as a challenge, I get a thrill out of it when I am able to overcome a problem,

I rather have other people adapt to me than me having to change my routine,

I'm tolerant to other people having bad days,

I feel unbearably overburdened from situations in my life,

I feel burdened from certain situations in my life,

Currently I feel stressed out,

I am active and manage my life without being overly stressed,

I feel burnt out,

Traumatic Stress:

I am bothered by things I cannot do anything about (like lowered morals, increased injustice, crimes, bad news or war in other places in the world)

The above intrudes on my mind several times a day

I feel angry about the above (world events)

I am bothered by injustice at work daily, or several times a week (circle: injustice occurs with me, or with others or both)

Bad events in the world just drains my energy

I try to avoid situations or reminders of bad things.

Thoughts or pictures intrude on my mind about bad things or injustice several times a day.

My feelings are numb about bad things or injustice,

Do you watch the TV news regularly, or listen to it on the radio? (Yes, I seek out to watch/hear the News several times a day, once a day, I watch it/listen to it if it happens to go on while I watch TV/listen to the radio, I purposefully try to avoid the News, I almost never watch/listen to the News),

If in a public place somebody walks behind me.. (a) I get nervous/suspicious so I let the other person pass me, (b) I keep an eye on the person behind me, but I keep walking, (a) It does not bother me/or it does not happening to me,

If a stressful or traumatic event comes to my mind, it makes me to want to have a drink, to smoke, or to eat, or just makes me angry or numb, (yes/no—usually true, or not true),

I try to avoid certain thoughts that remind me of a frightening experience I had or I have seen,

When I am watching news about violent local or worldwide events I have thoughts about revenge,

After watching terrible news on the TV, I have sad or disturbing thoughts, flashbacks or dreams about these events later on,

After watching news about violence, I feel being violated,

I have had discriminative or traumatic experiences in life,

I was bullied as a child,

I have happy thoughts after watching the news on the TV, (validity scale)

Anxiety:

I feel restless,

I get nervous,

I worry a lot,

I am afraid of many things.

I worry about what other people say or think about me.

I worry about the future,

I have a dooming feeling about the future,

I catch myself fantasizing/daydreaming of catastrophes or bad things happening in the future,

Somatization:

I have problems with physical illness or disability (circle: doctors cannot diagnose/brush off my problem; my illness interferes with my life; it incapacitates me; I'm under active treatment/or get disability for my physical problem),

I have physical pain, or aches,

I need a check up or continued care for stomach or intestinal problems,

I get headaches, stomach aches, or sickness quite often,

I have frequent thoughts that my body is disfigured or not perfect,

I have frequent thoughts that I may be ill, or that my body/organs are not functioning well,

I need others to take care of me, help me, or pay attention to me,

I think of seeking frequent medical attention to see if my body is working right,

I keep thinking of certain past situations in my life,

I feel restless,

I have frequent heart pounding for no reason at all,

I frequently get short of breath or sweating for no apparent reasons,

Guilt:

I feel inadequate,

I feel worthless,

I'm a failure,

I feel guilt without a reason,

I put myself down lately,

Cognitive distortion:

I'm a failure

Nobody likes me

I blame others a lot. I like to analyze situations to come up with the best possible solution for the problems, (−)

Obstacles are only challenges awaiting for me to solve them. (exactly true, moderately true, barely true, not true at all) (−)

I always take time to carefully weigh every possible outcome of a problem before making a decision on how to solve it. (−)

There are always many different ways to solve a problem (−)

I see the solution to overwhelming problems that they should be broken down to manageable components. (exactly true, moderately true, barely true, not true at all) (−)

I easily give up or run away form problems.

I feel that there is no point in trying to get help for my feelings/problems, as nothing would help anyway,

I tend to run away from problems in my life rather than trying for a solution, as I feel I cannot conquer the problems anyway,

I am a procrastinator, I like to postpone tasks in my life,

Unjust, resentment:

I often think of bad things or injustice even when I do not mean to.

I work too hard without being appreciated,

I have disillusionment or resentment associated with my work,

I have disillusionment or resentment associated with my family life,

I have serious resentments in my life,

Interpersonal sensitivity:

Do you feel sensitive?

I'm sensitive to critics, so I don't talk about my problems.

I'm a sensitive person, my feelings get hurt easily.

If I am put charge of organizing an event and I forgot about it I would.

(a) accept responsibility for my own action,

(b) find excuses about it,

(c) try to blame it on others,

(d) in no way it could be my fault, so it would have to be someone else goofing up

(e) why do you even think that it could be my mistake?

(intense/stormy relationships/ups and downs)

I'm a sensitive person,

I often take bad things in life personally,

I'm temperamental,

Helplessness hopelessness, (H/H,):

My situation is hopeless,

I do not feel helpless (−),

I feel that there is no point in trying to get help for my feelings/problems, as nothing would help anyway,

Optimism (scored negatively as being protective against depression and helplessness/hopelessness) (−):

If I have hit the bottom, things can only turn to the better. (exactly true, moderately true, barely true, not true at all)

One should never let his/her hopes down, that is what that leads to success. (exactly true, moderately true, barely true, not true at all)

Bad things in life are only temporary and do not last for ever. (exactly true, moderately true, barely true, not true at all)

I am optimistic about the future,

I can stay optimistic even in difficult times,

Withdrawal:

I'm lonely,

I feel lonely,

Lately, I'd like to be by myself, withdrawn, and not to get involved with others,

I like to keep my problems to myself rather than talking about it.

I often feel bored,

Do you make eye contact with others when you communicate?

More times than not I feel estranged from others,

Depression/Mood:

I'm satisfied with my life pretty much (−)

Are you generally warm to others? (−)

Dou you generally have peaceful feeling to others? (wishing others good things versus feeling revenge or anger), (−)

Would you have that you have a turbulent, unhappy, or balanced, successful life? (circle all that applies),

How many hours a day do you watch TV in average? None, 1, 2-3, 4-5, 6-8, 9-10, more than 10 hrs a day),

Do you mute the TV during advertisement?—or use TiVo of find other ways to avoid TV ads? Always, most often, half the time, rarely, never,

How much are you able to experience joy from simple things in life (like nature, music, sharing others joy)? (Not at all, a little, a moderate amount, very much, an extreme amount,),

I have an inner peace and harmony, (−)

I'm generally satisfied about my life, (−)

I have positive feelings in my life, (−)

I feel depressed or sad,

Interest:

I'm no longer enjoying the activities that you used to,

I have lost interest in sex,

I no longer keep up with my hobbies,

Things that gave me enjoyment, no longer interest me,

Concentration:

I'm hesitant, have difficulty making up my mind.

It is hard for me to keep my mind on my work.

I have a good attention, and I usually finish the things I start, (−)

I have been thinking been much slower lately,

I have trouble concentrating,

I have difficulty making decisions,

Appetite:

I seem to lost my appetite, or having trouble eating,

I'm eating too much lately,

I have lost some weight (or gained)

Psychomotorium:

I have noticed, or others told me, that I talked or moved more slowly, than it is normal for me,

I feel restless,

Self-esteem:

Some people like me or at least I like myself. (−)

I have confidence in myself, (−)

I feel worthless,

I'm a failure,

Relationships, Quality of life measures (QOL), (see also under stress):

I have problems with relationships—Circle all that applies: (romantic, or family/friends, or at work); (cannot initiate, maintain good relationships, do not have support),

There are problems with my living conditions (circle: eviction risk, financial strains, the rooms are a “mess”/unkempt/untidy)

Dou you generally interrupt others when talking? Dou you usually stay within the topic the other person raises, or tend to ignore it, not to reply or yet to switch to another topic and talk about your own agenda? (circle all that applies),

I am often putting off or “burying” different tasks or chores rather than prioritizing or not taking on the tasks I don't absolutely have to.

I am often behind schedule and procrastinate.

I seek out others advice and support when I have a problem. (−)

When I need it there is always someone there who listens to me. (−)

I feel accepted, (−)

How is your satisfaction in the following areas in your life?

Material things, you own?

Your relationships?

Your health, or your immediate family's health?

Your carrier/job/achievements?

Your safety, and/or the value system/moral in your immediate surrounding?

Are you in a positive balance/satisfied, average or withdrawal/unhappy state?

There is always a special person in my life on whom I can count on, (−)

I can talk about my problems to my family or friends. (−)

I have positive feelings in my life most every day, (−)

I'm in positive balance with good feelings in my life, (−)

Insight/therapeutic alliance:

Dou you feel you need to get psychiatric help?

Dou you feel you can manage your problems on your own?

Are you afraid ofjudgment or prejudice against depression or mental illness?

Is this fear strong enough that you would rather “fight” your symptoms than take medication, or get therapy?

Are you willing to take and continue to take psychiatric medication when prescribed by a doctor? Would you discontinue medication on your own rather than discuss it ahead of time with your doctor?

How much do you feel/think that therapy/treatment will help you and improve your life? Not at all, not too much, somewhat, I am confident, a great deal,

How much distress do you experience during your visits/assessment? (Not at all, only occasional mild one, significant, a great deal, almost intolerable or intolerable,)

How much hope do you have for improvement? (Not at all, somewhat, I am confident, a great deal, I feel excitement,)

The following items (questions) are also to be scored and/or relied on heavily:

(these questions may be asked after the test):

1, If I could change, or greatly improve any of my depressive symptoms I would first change (circle only one—you will have a chance to circle another symptom in the next question):

Decreased sleep, Decreased interest in pleasurable activities, Guilt, Decreased energy, Decreased concentration, Decreased appetite, Psychomotor retardation (being slow), Suicidal ideation, Anxiety, Somatic symptoms/pains and aches, Rumination/disturbing repetitive thoughts, Anger outbursts/impulsivity/hostility/violence, Cognitive distortion/jumping to conclusions/global thinking, Cognitive deficit/impairment, Social withdrawal, Helplessness/hopelessness, Stressors, or traumatic stress, Other symptoms/observed signs, Unjust/resentment, My sensitivity, Withdrawal, The depressed mood by itself, Self-esteem, Relationships, Balance on different aspects/quality of life,

2, If I could change, or greatly improve another of my depressive symptoms I would than change (circle only one—you will have a chance to circle another symptom in the next question):

Decreased sleep, Decreased interest in pleasurable activities, Guilt, Decreased energy, Decreased concentration, Decreased appetite, Psychomotor retardation (being slow), Suicidal ideation, Anxiety, Somatic symptoms/pains and aches, Rumination/disturbing repetitive thoughts, Anger outbursts/impulsivity/hostility/violence, Cognitive distortion/jumping to conclusions/global thinking, Cognitive deficit/impairment, Social withdrawal, Helplessness/hopelessness, Stressors, or traumatic stress, Other symptoms/observed signs, Unjust/resentment, My sensitivity, Withdrawal, The depressed mood by itself, Self-esteem, Relationships, Balance on different aspects/quality of life,

3, If I could have a third wish to change or greatly improve another depressive symptoms which would make a difference in my life, that would be (circle only one):

Decreased sleep, Decreased interest in pleasurable activities, Guilt, Decreased energy, Decreased concentration, Decreased appetite, Psychomotor retardation (being slow), Suicidal ideation, Anxiety, Somatic symptoms/pains and aches, Rumination/disturbing repetitive thoughts, Anger outbursts/impulsivity/hostility/violence, Cognitive distortion/jumping to conclusions/global thinking, Cognitive deficit/impairment, Social withdrawal, Helplessness/hopelessness, Stressors, or traumatic stress, Other symptoms/observed signs, Unjust/resentment, My sensitivity, Withdrawal, The depressed mood by itself, Self-esteem, Relationships, Balance on different aspects/quality of life,

4, I function in life at my baseline (as I usually did before becoming depressed)—circle one:

Yes at the same level, Somewhat less, Significantly less, Greatly incapacitated, Somewhat better (than my baseline before the illness), Moderately better (than my baseline before the illness), Significantly better (than my baseline before the illness), Feel really great, in harmony, better than ever (much much better than at my baseline before the illness),

5, Compared to when I started treatment for my depressive symptoms, (or if applicable compared to at my last visit) I function in life—circle one:

At the same level, Somewhat less, Significantly less, Much worse/greatly incapacitated, Somewhat better, Moderately better (than my baseline before the illness), Significantly better, Feel really great, in harmony, better than ever/much much better than before.

### EXAMPLE 2

In the first hypothetical case a 35 year old male patient comes to the Family doctor with vague somatic complaints. Upon examination no physical problems are found, but in according to DSM the diagnosis of MDD is made. No psychosis or delusions are present. There is no history of elevated mood or substance abuse, and the Family doctor does not make any Axis I or II diagnosis except for nicotine dependence. History reveals that this patient had been on an adequate dose of an SSRI two years ago, but had discontinued it within 3 months without telling to his previous doctor as he “did not feel any sudden or important change in his life, so he decided he did not need it”. More accurate assessment with the “extended” symptom list reveals self-defeating behaviors (smoking, overeating), disorganized work pattern, ruminative thinking, sleep problem, anger at work and home with impulsive acing out (slamming doors, punching walls, yelling). This all further took tool on the patient's relationships, with subsequently increased anxiety. His wife had mentioned the idea of divorce, and the patient had lost his job due to his acting out at work. Instead of looking at job advertisements, he was withdrawn and said to himself “nobody would hire me anyway” (cognitive distortion, and low self esteem). However he had maintained his hobbies. He denied suicidal ideation. The Family doctor prescribes an antidepressant-(low dose) antipsychotic medication combination (in part because the FDA is considered a black box warning on antidepressants for potential worsening the depression and the risk of causing suicide in children,—and that risk had affected the prescribing pattern in adults) and in part also because of him keeping up with the literature and relying on the knowledge of our above description of the “extended” depressive symptom list as well as the “pseudo-placebo” effect of various medications. In addition he also prescribes zolpidem for sleep for 10 days. Than the patient comes for follow up, and his symptoms are markedly improved due to the various symptoms this medication combination was targeting. The patient's expectation for further improvement was also positive (unlike the previous time he was on a single antidepressant). Further assessment later (after continued improvement) revealed the resolution of anger/impulsive/acting-out, the marked improvement in hopelessness and helplessness, cognitive distortions, rumination and sleep, along with the other depressive symptoms. The patient was also getting marriage therapy with reconciliation with his wife, and he had find a job and his quality of life had markedly improved. The Family doctor later presents this as a case report at a scientific meeting, with feedback from his colleagues. He was praised for choosing this medication combination as for the prevention of suicide, since another study from the same conference supported starting treatment with this (antidepressant-low dose atypical antipsychotic) combination having a more robust effect. The feedback from peers also pointed out that we do not know that who in the group would be adversely affected by suicide, and therefore the most effective treatment—with this combination—should be used for initial treatment as a rule, and emerging practice guideline.

### EXAMPLE 3

In this hypothetical example a film is made about the “clinical neuroplasticity” explanation of depression also using the “invisible mitt” expression, and the metaphor on the practice equivalent of “turning of dominos” as seen in the rehabilitation of stroke victims. A large employer with its own healthcare system in one regional part of the US sponsors the making of this film and the repeated showing of this film at the local PBS station, but it is only shown at this geographic region. The large employer/healthcare system also arranges that this video in an abbreviated form is also shown in the doctors' waiting room. (the video is also available in it's entire length along with patient educational material in the patient libraries). They also educate their own healthcare providers with this and other techniques of how to increase treatment adherence. In addition they are utilizing our methods of diagnosing/testing depression with the “extended symptom list” and our method of selecting medication or medication combinations relying on the knowledge of the “pseudo-placebo” effect of medications. Their survey show that the patients are more receptive to accept and adhere to treatment, and that the percentage of TRD decreased. A postgraduate student writes her dissertation on the data collected in comparison to other geographical regions, and finds that the expenses of healthcare and lost productivity at work arising from depression had markedly decreased. Additional findings reveal that the staff turnover decreases at that large employer/healthcare provider. An accidental finding requiring further studies finds that the illicit drug use and other self defeating behaviors (like smoking and overeating) also decreases.

It will be apparent to those skilled in the art that various modifications and variations can be made in the methods of the present invention without departing from the spirit or scope of the invention. Thus, it is intended that the present invention include modifications and variations that are within the scope of the appended claims and their equivalents.

### REFERENCES

1. WO 2004/010932 A2 Peter Migaly COMBINATION THERAPY FOR DEPRESSION, PREVENTION OF SUICIDE, AND VARIOUS MEDICAL AND PSYCHIATRIC CONDITIONS
2. U.S. patent application Ser. No. 10/627,358 Peter Migaly COMBINATION THERAPY FOR DEPRESSION, PREVENTION OF SUICIDE, AND VARIOU.S. MEDICAL AND PSYCHIATRIC CONDITIONS
3. 60319436 Peter Migaly New approaches to the treatment, assessment and research of depression
4. WO 99/58130 New drug combinations of a NARI, preferably reboxitene, and pindolol.
5. US 2002/0040041 A1 New drug combinations ((reboxitene, and pindolol.))
6. US 2002/0016325 A1 New drug combinations ((reboxitene, and pindolol.))
7. U.S. Pat. No. 6,322,503 B1 Method of diagnosing, tracking, and treating depression
8. WO 99/61027 May 1998, Combination therapy for treatment of refractory depression (Eli Lilly)
9. EP 0 958 824 A3 Olanzapine (Zyprexa) in combination with fluoxetine (Prozac), lithium, or anticonvulsant therapy of refractory depression 05. 1998.
10. U.S. Pat. No. 4,310,524 April, 1980, TCA composition and method for rapid onset antidepressant therapy.
11. U.S. Pat. No. 6,066,643 Potentiation of pharmaceuticals. ((antidepressants and atypical antidepressants))
12. U.S. Pat. No. 5,589,512 Methods for treating borderline personality disorder with serotonin reuptake blockers.
13. U.S. Pat. No. 6,159,963 Meathods for treating substance abuse ((about olanzapine))
14. Atmaca, A., et al. Quetiapine augmentation in patients with treatment resistant obsessive-compulsive disorder: a single-blind, placebo-controlled study. International Clinical Psychopharmacology, 2002, 17:115-119.
15. Berman, I., et al. The effect of novel antipsychotics on cognitive function. Psychiatric Annals. 29:11, 1999, 643-646.
16. Blier, P., et al. Putative mechanism of action of antidepressant drugs in affective and anxiety disorders and pain. J. Psychiatry Neurosci. 2001;26(1), 37-43.
17. Blier P. How many good antidepressant medications have we missed? Rev Psychiatr Neurosci 2004, 29(4) 248-49.
18. Blumenthal, J. A., et al. Effects of exercise training on older patients with major depression. Arch Intern Med. 1999, 159:2349-2356.
19. Bultema, J. Healing process for the multidisciplinary team: Recovering post-inpatient suicide. Journal of Psychosocial nursing. 1994, 32(2), 19-24.
20. Carlat, D. J. The Psychiatric Review of Symptoms: A Screening Tool for Family Physicians American Family Physicians Nov. 1, 1998 and http://www.aafp.org/afp/98 1101 ap/carlat.html (accessed Jan. 2, 2005).
21. Clark, D. A., Back, A. T. Scientific foundations of cognitive theory and therapy of depression. John Wiley, New York, 1999, page 52.
22. Colcombe S J, Erickson K I, Raz N, Webb A G, Cohen N J, McAuley E, Kramer A F. Aerobic fitness reduces brain tissue loss in aging humans. J Gerontol A Biol Sci Med Sci. 2003 58(2) 176-80.
23. Collaborative Working Group on Clinical Trial Evaluations. (1998 a), Evaluating the effects of antipsychotics on cognition in schizophrenia. J Clin Psychiatry, 1998:59 (suppl 12), 35-40.
24. Collaborative Working Group on Clinical Trial Evaluations. (1998 b), Atypical antipsychotics for treatment of depression in schizophrenia and affective disorders. J Clin Psychiatry, 1998:59 (suppl 12), 41-45.
25. Collaborative Working Group on Clinical Trial Evaluations. (1998 c), Treatment of special populations with the atypical antipsychotics. J Clin Psychiatry, 1998:59 (suppl 12), 46-52.
26. Cooper C. (1995 a) Psychiatric stress debriefing. Alleviating the impact of patient suicide and assault. Journal of Psychosocial Nursing. 1995, 33(5), 21-25.
27. Cooper C. (1995 b). Patient suicide and assault. Their impact on psychiatric hospital staff. Journal of Psychosocial Nursing. 1995, 33(6), 26-29.
28. Coull, J. T. et al Noradrenergically mediated plasticity in human attentional neuronal network. Neuroimage 1999 10(6) 705-15.
29. Cross-National Collaborative Group. The changing rate of major depression. JAMA, 268:21, 1992. 3098-3105.
30. Currier, G. W. Atypical antipsychotic medications in the psychiatric emergency service. J Clin Psychiatry, 2000(suppl 14) 21-26.
31. Deme, L. (Some aspects of children's linguistic education.) [in Hungarian] Tiszatáj (Szeged), 29, 1975, 16-21.
32. Deme, L. Personal communication. Kaptalanfired, Hungary.
33. Demyttenaere, K. et al. Compliance with antidepressants in a primary care setting, 1: Beyond lack of efficacy and adverse events. J Clin. Psychiatry, 2001; 62 (suppl 22) 30-33.
34. Depression and other mood disorders in Southwestern Pennsylvania
35. DSM-IV-TR Diagnostic and Statistical Manual of Mental Disorders Fourth Edition Text Revision. American Psychiatric Association Washington DC 2000.
36. Duman, R. S., et al. Role of gene expression in stress and drug-induced neural plasticity. TEN (The Economics of Neuroscience), 2000;2(4), 53-70.
37. Duman. R. S. et al. Neural plasticity to stress and antidepressant treatment. Biol. Psychiatry 1999, 46: 1181-1191.
38. Eisenberg L. TEN 2000, 2(4) 47-52
39. Forster P. Accurate assessment of short term suicide in a crisis. Psychiatric Annals 24:11, 1994 571-578.
40. George, M. S., et al. Activation studies in mood disorders. Psychiatric Annals, 24:12, 1994, 648-652.
41. Glazer, W. M., Formulary decisions and health economics. J. Clin Psychiatry, 1998; 59 (suppl 19), 23-29.
42. Grad. O. T. et al. Suicide of a patient: Gender differences in bereavement reactions of therapists. Suicide and Life-Threatening Behavior. 27(4), 1997, 379-386.
43. Gruber, A. J., et al. The management of treatment-resistant depression in disorders on the interface of psychiatry and medicine. The Psychiatric Clinics of North America, 19:2, 1996, 351-369.
44. Haney, C., et al “Interpersonal dynamics in a simulated prison”. International Journal of Criminology and Penology, 1973, 1; 69-97.
45. Harvey, J. A. Role of the serotonin 5-HT2A receptor in learning. Learning and Memory 2003 10(5) 355-62.
46. Hughes, H. D. Pharmacoligic management of the chronically aggressive psychiatric patients. Psychiatric Annals 28:7, 367-370, 1998.
47. Kamholz, B. A. et al. Management of treatment resistance in the depressed geriatric patient. The Psychiatric Clinics of North America. 19:2, 1996, 269-286.
48. Kaplan, H. I. et al. Kaplan and Sadock's Synopsis of Psychiatry (8^{th} edition) 1998 Williams & Wilkins, Baltimore
49. Keck, P. E., et al (2000 b) The efficacy of atypical antipsychotics in the treatment of depressive symptoms, hostility, and suicidality in patients with schizophrenia. J. din Psychiatry. 2000;61, (suppl. 3) 4-9.
50. Keck, P. E., et al (2000 b) The efficacy of atypical antipsychotics in the treatment of depressive symptoms, hostility, and suicidality in patients with schizophrenia. J. din Psychiatry. 2000;61, (suppl. 3) 4-9.
51. Keefe, R. S., et al. Do novel antipsychotics improve cognition? A report of a meta-analysis. Psychiatric Annals. 29:11, 1999, 623-629.
52. Ketter, T. A., et al. Primary mood disorders: structural and resting functional studies. Psychiatric Annals, 24:12, 1994, 637-642.
53. Khan, A., et al. Severity of depression and response to antidepressants and placebo: an analysis of the Food and Drug Administration database. Journal of Clinical Psychopharmacology, 22:1, 2002, 40-45.
54. Kirmayer, L. J. Cultural variations in the clinical presentation of depression and anxiety: implications for diagnosis and treatment. J. Clin. Psychiatry, 62: (Suppl. 13) 22-28. 2001.
55. Kirmayer, L. J., et al. Somatization and the recognition of depression and anxiety in primary care. Am. J. Psychiatry, 1993; 150: 734-741.
56. Kirsch, I. Are drug and placebo effects in depression additive? Biol Psychiatry 2000;47:733-735.
57. Knieper, A. J. The survivor's grief and recovery. Suicide and Life-Threatening Behavior. 29(4), 1999, 353-364.
58. Koh, K. B. et al. Predominance of anger in depressive disorders compared with anxiety disorders and somatoform disorders. J. Clin Psychiatry 2002;63:486-489.
59. Lee, A. L., et al. Stress and depression: possible links to neuron death in the hippocampus. Bipolar Disorders 2002, 4: 117-128.
60. Lubin, B., State Trait Depression Adjective Check List (ST-DACL) EdITS 2002
61. Lynch, M. E. Antidepressants as analgesics: a review of randomized controlled trials. J. Psychiatry Neurosci. 2001;26(1), 30-6.
62. Lyness J. M. et al. Ruminative thinking in older inpatients with major depression. J. Affective Disorders 46, (1997), 273-277.
63. Malberg J. E. Implications of adult hippocampal neurogenesis in antidepressant action. Rev Psychiatr Neurosci 2004 29, (3) 196-205.
64. Meichenbaum D. et al Facilitating Treatment Adherence Plenum Press New York, 1987.
65. Melzack, R. The puzzle of pain. Basic Books, New York, 1973.
66. Meneses A. A pharmacological analysis of an associative learning task: 5-HT1 to 5-HT7 receptor subtypes function on a Pavlovian/instrumental autoshaped memory. Learn Mem 2003 10(5) 363-72.
67. Mental health resources by topic: http://www.library.adelaide.edu.au/guide/med/menthealth/scales.html (accessed Aug. 6, 2004)
68. Miller, D. S. et al. Comparative efficacy of typical and atypical antipsychotics as add-on therapy to mood stabilizers in the treatment of acute mania. J. Clin. Psychiatry, 62:12, 975-980. 2001.
69. Mohr, N., et al,: Quetiapine augmentation of serotonin reuptake inhibitors in obsessive-compulsive disorder. International Clinical Psychopharmacology 2002; 17 (January): 37-40.
70. Mullein J. et al. (ed) Manual of Rating Scales for the assessment of mood disorders (2004 AstraZeneca)
71. News & Notes: Cost of depression estimated at nearly $44 billion; time lost from work accounts for largest share. Hospital and Community Psychiatry. 1994, 45:(1), 85.
72. Nezu, A. M. et al Practitioner's guide to empirically based measures of depression. Kluwer Academic/Plenum Publishers New York 2000.
73. Nurse J. et al. Influence of environmental factors on mental health within prisons: Focus group study. Br Med J 2003, 327;480-484.
74. O'Connor, M., et al. Adding risperidone to selective serotonin reuptake inhibitor improves chronic depression. J. Clin. Psychopharmacol. 18:1, 89-91, 1998.
75. O'Connor, R. Active treatment of depression. WW Norton & Company, New York, 2001.
76. PBS 1940's House
77. PBS Amazing Grace with Bill Moyers.
78. PBS Frontier House
79. Pincus, H. A. et al. The societal cost of chronic major depression. J. Clin Psychiatry, 2001, 62 (suppl 6). 5-9.
80. Purdon, S. E., et al. Neurological change in patients with schizophrenia after treatment with quetiapine or haloperidol. J. Psychiatry Neurosci. 2001;26(2):137-49.
81. Restak, R., The secret life of the brain. Dana Press and Joseph Henry Press 2001.
82. Rihmer, Z. Can better recognition and treatment of depression reduce suicide rates? A brief review. Eur. Psychiatry 2001, 16: 406-9.
83. Rosenzweig, E. S. et al Impact of aging on hippocampal function: plasticity, network dynamics, and cognition. Prog Neurobiol 2003 69(3) 143-79.
84. Rossi E.
85. Rubin, E. et al. Brain imaging studies of antidepressant treatments. Psychiatric Annals, 24:12, 1994, 653-658.
86. Rush, A. J. The use of placebos in unipolar major depression: the current status. Biol Psychiatry, 47: 2000, 745-747.
87. Russo-Neustadt, A., et al. Exercise, antidepressant medications, and enhanced brain derived neurotrophic factor expression. Neuropsychopharmacology, 1999, 21:5, 679-682.
88. Sajatovic, M. et al. Quetiapine alone and added to mood stabilizers for serious mood disorders. J. Clin. Psychiatry, 62:9, 728-732. 2001.
89. Salin-Pascual, R. J. et al. Olanzapine acute administration in schizophrenic patients increases delta sleep and sleep efficiency. Biol. Psychiatry, 1999, 46:141-143.
90. Satel, S. L., et al. Stimulants in the treatment of depression: a critical overview. J. Clin Psychiatry, 50:241-249.
91. Shelton, C. R., et al. (2001 a) A novel augmentation strategy for treating resistant major depression. Am J Psychiatry 158:1, January 2001 131-134.
92. Simon, R. I., Suicide risk assessment in Managed Care settings. Primary Psychiatry, April 2002; Vol 9. NO.4. 42-49.
93. Singleton, S. S. Depression and quality of life: a patient's perspective. J. Clin. Psychiatry, 62; (suppl 26), 2001, p22.
94. Spitzer, M. The mind within the net. Models of learning, thinking, and acting. A Bradford Book, Cambridge, 1999.
95. Stahl, S. M. Does depression hurt? J. Clin. Psychiatry, 63:4, 273-274. 2002.
96. Stajatovic, M. et al. (eds): Manual of rating scales for the assessment of serious mental illness. 1999. Zeneca, Inc
97. Szadoczky, E. [The clinical manifestations of mood disorders]=A hangulatzavarok klinikai megjelenese (in Hungarian); 26-58. In: [Mood disorders]=Hangulatzavarok (in Hungarian), Szadoczky, E., Rihmer Z. (eds.) Medicina, Budapest, 2001.
98. Thase, M. (2002 a): Comparing the methods used to compare antidepressants. Psychopharmacology Bulletin: Spring 2002-Vol. 36, Suppl. 14-17
99. Thase, M. (2002 b): What role do atypical antipsychotic drugs have in treatment-resistant depression? J. Cin. Psychiatry 63:2, February 2002, 95-103.
100. Thase, M. (2002 c): Studiing new antidepressants: if there were a light at the end of the tunnel, could we see it? J. Cin. Psychiatry 63 (suppl2) 2002, 24-28.
101. Trudeau, L-E. Glutamate co-transmission as an emerging concept in monoamine neuron function. Rev Psychiatr Neurosci 2004, 29(4) 269-310.
102. Tsai, S Y. M., et al.: Risk factors for completed suicide in bipolar disorder. J. Clin Psychiatry. 2002:63:469-476.
103. Twerski A. tape
104. Vaillant, G. E. 1993. The Wisdom of Ego. Harvard University Press Cambridge MA 1993.
105. Van Kammen, D. P. et al. Dopamine receptor antagonists. Page 1990. In: Kaplan, H. I. et al. (eds) Comprehensive textbook of psychiatry. Williams & Wilkins, Baltimore, 1995.
106. Vogel, G. New brain cells prompt New theory of depression. Science. 290, 2000, 258-259.
107. Wallenstein G. V. et al. Effectiveness of community-based screening for depression. Psychiatric Times June 2004 pp 80, 82.
108. Wallenstein G. V. et al. Effectiveness of community-based screening for depression. Psychiatric Times June 2004 pp 80, 82.
109. Weissman, M. M. Social functioning and the treatment of depression. J. Clin Psychiatry, 2000;61 (suppl 1), 33-38.
110. Willner, P. The mesolimbic dopamine system as a target for rapid antidepressant action. International Clinical Psychopharmacology, 1997, 12 (suppl 3):S7-S14.
111. Wittchen, H-U., et al. Lifetime risk of depression. British Journal of Psychiatry, 165 (suppl 26), 1994, 16-22.
112. Yale-Brown Obsessive Compulsive Scale (Y-BOCS)
113. Yapko tape
114. Yardley, K. M. (1982 a) On distinguishing role plays from conventional methodologies. J. Theory Soc. Behaviour. 12; 1982, 125-139.
115. Yardley, K. M. (1982 b) On engaging actors in as-if experiments. Journal for the Theory of Social Behaviour. 12(3) 1982, 291-304.
116. Yatham, L. N. The role of novel antipsychotics in bipolar disorders. J. Clin Psychiatry; 63 (suppl. 3) 10-14. 2002.
117. Zimmerman, M., et al: Major depressive disorder and Axis I diagnostic comorbidity. J. Clin. Psychiatry 63:3, 2002; 187-183.
118. Zuckerman, M., Lubin, B, Multiple Affect Adjective Check List EdITS 1965.

The invention claimed is:

1. A method of diagnosing and treating a depressive disorder in a patient, comprising challenging the traditional dogma of the Diagnostic and Statistical Manual IV-TR, which has set the current standard of care with it's criteria sets, as being only a differential diagnostic manual primarily useful for differentiating depression from other mental disorders; challenging that the Diagnostic and Statistical Manual IV-TR's limited number of depressive symptoms in it's criteria set is accurate and sensitive enough to diagnose, monitor and test for depressive symptoms and accurate and sensitive enough to test for remission of depressive symptoms, said limited number of depressive symptoms consisting of depressed mood, decreased sleep, decreased interest, guilt, decreased energy, decreased concentration, decreased appetite, psychomotor retardation and suicidal ideation; and challenging that the Hamilton Depression Rating Scale or equivalent scale(s), which rating scale relies on the Diagnostic and Statistical Manual IV's limited number of depressive symptom list, is accurate and sensitive enough to diagnose, monitor and test for depressive symptoms and accurate and sensitive enough to test for remission of depressive symptoms, by providing an extended list of depressive symptoms that is accurate and sensitive enough and that can be systematically relied upon to diagnose, test and monitor for depressive symptoms, said extended list selected from the group consisting of anxiety, somatic concerns/somatization/focusing on somatic symptoms, rumination/obsessiveness, anger/irritability/impulsivity/hostility/violence, cognitive distortion/global thinking, cognitive deficit/impairment, social withdrawal, helplessness/hopelessness, acute and chronic stressors and the patient's ability to cope with them and to problem solve, perception of unjust and resentment, indifference, sensitivity, and other symptoms and observed signs.

2. The method of claim 1, further comprising producing a pseudo-placebo effect that results in conditioning and expectation changes in the patient that are relied on and utilized to treat the depression in the patient, wherein the pseudo-placebo effect is comprised of targeting each or any of the of the depressive symptoms, which include the extended symptom list, and not only the general depressed mood itself, with psychological and/or pharmacological intervention.

3. The method of claim 1, wherein said method increases the effectiveness of the treatment of depression, provides a quicker response to treatment, provides a better chance to achieve full remission and increases the patients' quality of life, thus decreasing the risk of suicide.

4. The method of claim 1, wherein percentage of cases of treatment resistance depression and percentage of cases of depression only in partial remission are decreased in the population of patients afflicted with depression.

5. The method of claim 2, wherein examples for the pseudo-placebo effect utilizing a pharmacological treatment is selected from the group of antidepressants selected from the group consisting of serotonin reuptake inhibitors, selective norepinephrine reuptake inhibitors, combined action SSRI/SNRI, serotonin-2 antagonist/reuptake inhibitors, antidepressants with alpha-2 antagonism plus serotonin-2 and serotonin-3 antagonism, antidepressants with serotonin/norepinephrine/dopamine reuptake inhibition and antidepressants with norepinephrine and dopamine reuptake inhibition; and/or said antidepressants are selected from the group consisting of tricyclic antidepressants, tetracyclic antidepressants and MAOI inhibitors; and/or said antidepressants are selected from the group consisting of 5-HT-lalpha antagonists, 5-HT-1beta antagonists, 5-HT1A receptor agonists, 5-HT1A receptor agonists and antagonists, 5-HT2 receptor antagonists, viloxazine hydrochloride, dehydroepiandosterone, NMDA receptor antagonists, AMPA receptor potentiators, substance P antagonists/neurokinin-1 receptor antagonists, nonpeptide Substance P antagonists, neurokinin 2 antagonists, neurokinin 3 antagonists, corticotropin-releasing factor receptor antagonists, antiglucocorticoid medications, glucocorticoid receptor antagonists, cortisol blocking agents, nitric oxide synthesize inhibitors, inhibitors of phosphodiesterase, enkephalinase inhibitors, GABA-A receptor agonists, free radical trapping agents, atypical MAOI's, selective MAOI inhibitors, hormones, folinic acid, leucovorin, tramadol, tryptophan and pharmaceutically acceptable salts thereof; and/or said antidepressant includes clomipramine; and/or wherein the antidepressants are selected from the group consisting of fluoxetine, norfluoxetine, paroxetine, sertraline, fluvoxamine, citalopram, escitalopram, bupropion, nefazodone, mirtazapine, venlafaxine, duloxetine, milnacipran, reboxetine, zimelidine, indalpine, gepirone, femoxetine, alaproclate, duloxetine and pharmaceutically acceptable salts thereof; or wherein examples for the pseudo-placebo effect utilizing a pharmacological treatment includes atypical antipsychotics and/or dopamine system stabilizers, and/or antipsychotics selected from the group consisting of risperidone, quetiapene, olanzapine, ziprasidone and aripiprazole, wherein said atypical antipsychotics are selected from the group consisting of olanzapine, iloperidone, Org 5222, melperone, amperozide, SM-9018, JL-13 and pharmaceutically acceptable salts thereof; or wherein said antipsychotics are selected from the group consisting of perphenazine, trifluoperazine, zotepine, flupenthixol, amisulpride, and sulpiride; or wherein examples for the pseudo-placebo effect utilizing a pharmacological treatment includes anxiolytics selected from the group consisting of clonazepam, benzodiazepines, antipsychotics, atypical antipsychotics and/or dopamine system stabilizers; or wherein examples for the pseudo-placebo effect utilizing a pharmacological treatment includes medications targeting decreased or disturbed sleep with sleeping pills selected from the group consisting of zolpidem and benzodiazepines, as well as with targeting sleep problems through sleep hygiene, correcting sleep apnea with continuous positive airway pressure (CPAP), or correcting overweight; or wherein examples for the pseudo-placebo effect utilizing a pharmacological treatment includes medications targeting anger/violence/anxiety such as the beta blockers propranolol or pindolol; or wherein examples for the pseudo-placebo effect utilizing a pharmacological treatment includes medications targeting fatigue and tiredness, like modafinil, or wherein examples for the pseudo-placebo effect utilizing a pharmacological treatment includes medications targeting decreased energy, such as stimulants, or wherein examples for the pseudo-placebo effect utilizing a pharmacological treatment includes medications that target disorders selected from the group consisting of obsessiveness/rumination, cognitive distortions, jumping to conclusion, somatic symptoms and perceptual disturbance relating to somatic symptoms, and wherein examples for the pseudo-placebo effect utilizing a pharmacological treatment includes any of the pharmacological treatments or combinations thereof that are known to target these symptoms are utilized.

6. The method of claim 1, wherein a psychological test comprised of a psychometric instrument incorporates the extended symptom list that is systematically relied upon.

7. The method of claim 6, wherein the extended symptom list comprises at least five extended symptoms.

8. The method of claim 6, wherein the extended symptom list comprises at least six extended symptoms.

9. The method of claim 6, wherein the extended symptom list comprises at least seven extended symptoms.

10. The method of claim 6, wherein the extended symptom list comprises at least eight extended symptoms.

11. The method of claim 6, wherein the psychological test comprised of a psychometric instrument that is based on the more extended symptom list provides a more sensitive method to test and monitor depression than existing tests and provides a more practical way to better test antidepressant effectiveness and to test which antidepressant or combination thereof is more effective and/or quicker acting.

12. A method of increasing treatment adherence and decreasing resistance for seeking help in a depressed patient as well as decreasing or eliminating prejudice and the stigma against depression as a mental illness, comprising describing to the patient, as well as to health care providers and to the general public, about clinical studies that do not pertain directly to mental depression involving neuroplasticity of the brain, neurogenesis, and/or brain mapping, wherein that is linked to neuroplasticity in the brain and mental depression and/or brain mapping in depression with a description of a metaphor.

13. The method of claiml2, wherein the description comprises as if/role play experiments that cause depression or lift depression and/or environmental situations that could cause depression in anybody.

14. The method of claiml3, wherein the resolution of these environmental situations or as if/role play experiments are linked to the resolution of depressive symptoms with accompanied neuroplasticity.

15. The method of claiml2, wherein the linking to mental depression and neuroplasticity pertains to clinical neuroplasticity.

16. The method of claiml2, wherein the metaphors are selected from the group consisting of an “invisible mitt” being equivalent to restraining negative thought patterns/rumination with medications and/or with cognitive therapy; the metaphor of practice or practice makes a master, which is a metaphor of practicing the equivalent of turning of dominos in the physical therapy of stroke victims, which is equivalent to the practice of cognitive therapy; the metaphor of the need for practicing having optimistic thoughts, which can result from increasing positive expectations; the metaphor of that six hours a day practice was needed for stroke victims therefore in the treatment of depression one hour of practice or psychotherapy per week or even per day may not be enough to counter the old habit/depressive thinking, so the “invisible mitt/medication(s) and/or diligent homework practice is needed; the metaphor to explain of why it is so easy to relapse if one stops the invisible mitt/medication and/or the practice; and using the description and examples of pseudo-placebo conditioning/expectation changing effects of medications on depression to describe various treatment strategies/alternatives for the treatment of depression, or that why antidepressants have a large placebo effect.

17. The method of claim 16, wherein the metaphor may be used to explain the risk of relapse to depression if the medications are discontinued, or if the patterns of thoughts are shifted again from predominantly positive to negative/ruminative thoughts.

18. The method of claim 12, wherein the link is made to the patient, health care providers and to the general public that the hippocampal volumetric and/or morphologic changes seen in depression may result from the process of learning and/or memory because learning is involved in the process of mental depression, rather than resulting from the change of mood per se, and wherein this new way of understanding the reasons for hippocampal changes in depression fits in well with findings that the hippocampus is involved in learning and memory, which may guide pre-clinical research for new antidepressants.

19. A method of increasing treatment adherence and decreasing resistance for seeking help in a depressed patient, comprising:
meeting the client needs by the healthcare provider;
the healthcare provider changing his/her approach repeatedly so that the client's needs are met and a desired change occurs, wherein the patient perceives a gain, that the situation is right and has a readiness for change; that the timing for change is perceived as the right time, that the need for change being important and emergent enough be realized, that the relative easiness of the change or that the process of change can come with at least some enjoyment and satisfaction can be realized, and that the situation is adjusted so that the patient would have the tools and skills needed for the desired change.

20. The method of claim 19, further comprising:
describing to the patient new information in simple ways so as to make them feel as if they already knew that information, thus relating to the information and/or accepting the information as their own;
eliciting positive emotions expectations from the patient; validating the patient's feelings through universal human experiences; raising hope and/or increasing confidence in the patient;
giving the patient new, direct, personal experience that change is possible; and
acknowledging that rapid change is possible, wherein the patient, as well as health care providers and the general public are educated via marketing techniques of this method.

21. The method of claim 19, wherein the method can be used in cases other than depression, such as diagnostic categories where depression is often a co-morbid and/or underlying condition, and wherein the cases are selected from the group consisting of smoking-cessation, nicotine withdrawal, addiction, overweight/obesity/weight control, eating disorders, chronic pain, other mental disorders, and in other cases not related to depression.,

22. The method of claim 5, wherein the pseudo-placebo effects of the medication(s) and/or their increased effectiveness targeting more than one depressive symptoms protects against or remedies the development of tolerance towards antidepressant treatment, SSRI treatment, and/or wherein that prevents a paradoxical effect of the antidepressant treatment or SSRI treatment that sensitizes patients to depression, avoids or treats worsening of depression from the antidepressant treatment, or SSRI treatment, treats residual symptoms of depression, and/or decreases suicide and/or the risk of suicide.

23. The method of claim 22, wherein treatment is given as initial treatment or as soon as possible, or upon presentation to a physician or a health care provider for preventing suicide.

24. The method of claim 1 further comprising educating or marketing to the patients or to the public of treatment strategies and/or treatment alternatives as it pertains to the pseudo-placebo/conditioning effects of medications on the treatment of depression.

25. The method of claim 1 further comprising educating the patients or to the public on the misconception of the limited number of symptom list in Diagnostic and Statistical Manual IV-TR's criteria set and/or of treatment strategies and/or treatment alternatives arising from that as of targeting the depressive symptoms other than the mood, and utilizing the pseudo-placebo/conditioning effects of medications in the treatment of depression.

26. The method of claim 13 further comprising educating the patients or to the public that the clinical neuroplasticity explanation of depression may shift the focus off the neurotransmitter deficit explanation of depression, and/or the serotonin's direct effect on the mood, and shifting the focus to the importance of targeting the various depressive symptoms other than the mood.

27. A method of diagnosing and treating a depressive disorder in a patient comprising administering a psychopharmacological diagnostic/treatment/educational modality to the patient, comprising:
a) assessing the presence and severity of a constellation of symptoms in the patient;
b) administering in therapeutic amounts at least one or more psychoactive drugs to the patient, wherein the at least one or more psychoactive drugs are selected based on the results of (a) so as to target the symptoms that are present;
c) educating the patient as to the neuroplasticity response of the brain that is ubiquitous to any human, wherein said education reduces negative feelings of the patient;
d) meeting the patient's needs, wherein said meeting of the patient's needs increases positive feelings in the patient;
e) reassessing at a later time the presence and severity of the constellation of symptoms in the patient;
f) adjusting the therapeutic amounts of the at least one or more psychoactive drugs based on the results of (e);
g) administering the adjusted therapeutic amounts of the at least one or more psychoactive drugs to the patient; and
h) repeating steps (c) through (g) until the presence and severity of the constellation of symptoms have diminished to a subclinical level.

28. The method of claim 27, wherein the constellation of symptoms include the presence of at least five or more of the following symptoms from (a), wherein one of the symptoms is either depressed mood or loss of interest or pleasure, as well as the presence of five or more of the following symptoms from (b):
depressed mood; diminished interest or pleasure in all or almost all activities; significant weight loss when not dieting or weight gain, or decrease or increase in appetite; insomnia or hypersomnia; psychomotor agitation or retardation, fatigue or loss of energy; feelings of worthlessness or excessive or inappropriate guilt; diminished ability to think or concentrate, or indecisiveness; or suicidal ideation; and
anxiety, irritability, obsessiveness/rumination; anger, helplessness/hopelessness; impulsivity; hostility; violent behavior; resentment; excessive emotional sensitivity; indifference; cognitive distortion; or social withdrawal.

29. The method of claim 27, wherein the depressive disorder is selected from the group consisting of major depressive disorder, dysthymic disorder, dual depression, depressive disorder not otherwise specified, substance/alcohol induced mood disorder, postpartum depression and adjustment disorder with depressed mood.

30. The method of claim 27, wherein the psychoactive drug is an antidepressant drug.

31. The method of claim 30, wherein the antidepressant drug is selected from the group consisting of serotonin reuptake inhibitors, a selective norepinephrine reuptake inhibitors, combined action SSRI/SNRI, serotonin-2 antagonist/reuptake inhibitors, an antidepressant with alpha-2 antagonism plus serotonin-2 and serotonin-3 antagonism, an antidepressant with serotonin/norepinephrine/dopamine reuptake inhibition and an antidepressant with norepinephrine and dopamine reuptake inhibition.

32. The method of claim 27, wherein the psychoactive drug is an antipsychotic drug.

33. The method of claim 32, wherein the antipsychotic drug is selected from the group consisting of quetiapine, risperidone, ziprasidone, olanzapine, iloperidone, Org 5222, melperone, amperozide, SM-9018, JL-13, aripiprazole and pharmaceutically acceptable salts thereof.

34. The method of claim 32, wherein the antipsychotic drug is selected from the group consisting of perphenazine, trifluoperazine, zotepine, flupenthixol, amisulpride and sulpiride.

35. The method of claim 27, wherein the psychoactive drug is selected from the group consisting of clonazepaam, a benzodiazepine, zolpidem, propranolol, pindolol, modafinil and duloxetine.

36. The method of claim 27, wherein the negative feelings that are reduced in the patient are selected from the group consisting of self-blaming, shame, guilt, helplessness, hopelessness, unhappiness, melancholia, discouragement, sorrow, gloominess, miserableness, torment and feelings of being stigmatized because of the depressive disorder.

37. The method of claim 27, wherein the positive feelings that are increased in the patient are selected from the group consisting of hopefulness, satisfaction, expectation of successful changes, happiness, contentment, comfortableness, pleasure, joy, peace, harmony, cheerfulness, euphoria, ecstasy, delight, gratefulness and wellness.

38. The method of claim 27, wherein the patient's needs that are met are selected from the group consisting of unconditional acceptance, appreciation, praise, understanding, concern, patience and limit setting.

39. The method of claim 27, wherein educating the patient as to the neuroplasticity response of the brain improves compliance of the patient to treatment.

40. A method of producing a pseudo-placebo effect in a patient afflicted with a depressive disorder in order to produce rapid global improvement of symptoms and satisfaction in the patient, comprising:
targeting one or more specific symptoms of the patient which are known to respond quickly to drug administration; and
administering in therapeutic amounts at least one or more psychoactive drugs known to affect the one or more specific symptoms targeted, wherein alleviation of one or more symptoms elicits a conditioned reflex in the patient which results in a rapid global improvement of other symptoms of the patient, thus increasing patient satisfaction and further positive changes in the patient.

41. The method of claim 40, wherein the one or more symptoms of the patient are selected from the group consisting of anxiety; helplessness/hopelessness; insomnia;fatigue; obsessiveness/rumination; anger/violence; cognitive distortions and perceptual/somatic disturbance.

42. The method of claim 40, wherein the one or more psychoactive drugs include antidepressants, antipsychotics, anxiolytics, sedatives or beta blockers.

43. The method of claim 40, wherein anxiety is treated with antipsychotics, clonazepam or benzodiazepines; helplessness/hopelessness is treated with antipsychotics; insomnia is treated with sedatives, such as zolpidem or antipsychotics; anxiety is treated with anxiolytics; anger and violence is treated with antipsychotics, anger, violence and anxiety are treated with beta blockers, such as propranolol or pindolol; fatigue is treated with modifinil; obsessiveness/rumination is treated with SSRIs and/or a combination of antipsychotics/antidepressants; cognitive distortions are treated with antipsychotics; somatic/perceptual disturbance is treated with antipsychotics and/or antidepressants; and social withdrawal is treated with antidepressants and/or antipsychotics.

44. The method of claim 40, wherein the one or more of the psychoactive drugs are selected from the group consisting of clonazepam, a benzodiazepine, zolpidem, propranolol, pindolol, modafinil and duloxetine.

45. A method of improving patient compliance in the treatment of an already-diagnosed depressive disorder, comprising:
educating the patient as to the neuroplasticity response of the brain that is ubiquitous to any human, wherein said education reduces negative feelings of the patient;
assessing the patent for the reduction of negative feelings; and
promptly administering to the patient a psychoactive drug designed to address the patient's symptoms of the depressive disorder.

46. The method of claim 45, wherein the negative feelings that are reduced in the patient are selected from the group consisting of self-blaming, shame, guilt, helplessness, hopelessness, unhappiness, melancholia, discouragement, sorrow, gloominess, miserableness, torment and feelings of being stigmatized by others because of the depressive disorder.

47. The method of claim 45, wherein the psychoactive drug is an antidepressant drug.

48. The method of claim 47, wherein the antidepressant drug is selected from the group consisting of serotonin reuptake inhibitors, a selective norepinephrine reuptake inhibitors, combined action SSRI/SNRI, serotonin-2 antagonist/reuptake inhibitors, an antidepressant with alpha-2 antagonism plus serotonin-2 and serotonin-3 antagonism, an antidepressant with serotonin/norepinephrine/dopamine reuptake inhibition and an antidepressant with norepinephrine and dopamine reuptake inhibition.

49. The method of claim 45, wherein the psychoactive drug is an antipsychotic drug.

50. The method of claim 49, wherein the antipsychotic drug is selected from the group consisting of quetiapine, risperidone, ziprasidone, olanzapine, iloperidone, Org 5222, melperone, amperozide, SM-9018, JL-13, aripiprazole and pharmaceutically acceptable salts thereof.

51. The method of claim 49, wherein the antipsychotic drug is selected from the group consisting of perphenazine, trifluoperazine, zotepine, flupenthixol, amisulpride and sulpiride.

52. The method of claim 45, wherein the psychoactive drug is selected from the group consisting of clonazepam, a benzodiazepine, zolpidem, propranolol, pindolol, modafinil and duloxetine.
